(19) United States
(12) Patent Application Publication
(10) Pub. No.: US 2023/0105319 A1
(43) Pub. Date: Apr. 6, 2023

(54) PRODRUG INCORPORATED sgRNA SYNTHESIS

(71) Applicant: CRISPR Therapeutics AG, Zug (CH)

(72) Inventors: Rajat Das, Cambridge, MA (US); Ganapathy Sankaran, Cambridge, MA (US)

(21) Appl. No.: 17/934,796

(22) Filed: Sep. 23, 2022

Related U.S. Application Data
(60) Provisional application No. 63/248,272, filed on Sep. 24, 2021.

(51) Int. Cl. C12N 15/11; C12N 9/22

(52) U.S. Cl. CPC C12N 15/11; C12N 9/22; C12N 2310/321; C12N 2310/531; C12N 2310/3231; C12N 2310/315; C12N 2310/314; C12N 2310/20; C12N 2800/80; C12N 2310/346; C12N 2310/351

(57) ABSTRACT

Disclosed herein include guide RNA (gRNA), such as single gRNA (sgRNA), and compositions thereof, comprising 2′-O-methyldithiomethyl modified sugar moieties which can be reduced to 2′-O-methanethiol groups in the reducing environment of cells and then converted (e.g., spontaneously converted) to 2′-OH. The resultant gRNA can bind to and direct the activity of an RNA-guided endonuclease (e.g., Cas9).

### CROSS-REFERENCE TO RELATED APPLICATIONS

This application claims the benefit under 35 U.S.C. § 119(e) to U.S. Provisional Patent Application No. 63/248,272 filed on Sep. 24, 2021, the content of which is incorporated herein by reference in its entirety for all purposes.

### REFERENCE TO SEQUENCE LISTING

The present application is being filed along with a Sequence Listing in electronic format. The Sequence Listing is provided as a file entitled Sequence_Listing_80EM-341698-US, created Sep. 12, 2022, which is 21 kilobytes in size. The information in the electronic format of the Sequence Listing is incorporated herein by reference in its entirety.

### BACKGROUND

### Field

The present disclosure generally relates to the field of molecular biology and biotechnology, including the synthesis of guide RNA.

### Description of the Related Art

The targeting of DNA using the RNA-guided, DNA-targeting principle of CRISPR (Clustered Regularly Interspaced Short Palindromic Repeats)-Cas (CRISPR associated) systems has been widely used. CRISPR-Cas systems can be divided in two classes, with class 1 systems utilizing a complex of multiple Cas proteins (such as type I, III, and IV CRISPR-Cas systems) and class 2 systems utilizing a single Cas protein (such as type II, V, and VI CRISPR-Cas systems). Type II CRISPR-Cas-based systems have been used for genome editing, and require a Cas polypeptide or variant thereof guided by a customizable guide RNA (gRNA) for programmable DNA targeting.

Available approaches for the synthesis of gRNA include intracellular transcription of an exogenous plasmid and solid-phase synthesis using phosphoramidite chemistry. Direct chemical synthesis of gRNA allows for incorporation of chemical modifications that modulate gRNA-Cas binding, increase the chemical stability of the gRNA, decrease its immunogenicity, and reduce potential off-target effects (i.e., cleaving genomic DNA at undesired locations). There is a need for gRNA that is conditionally activatable, with increased chemical stability, decreased immunogenicity, and reduced potential off-target effects.

### SUMMARY

Disclosed herein include embodiments of an oligomeric compound. In some embodiments, an oligomeric compound comprises a modified oligonucleotide according to the following formula:

NₘₛNₘₛNₘₛNₜNₜNₜNₜNₜNₜNₜNₜNₜNₜNₜNₜNₜNₜNₜNₜNₜ

GₜUₜUₜUₜUₜAₜGₜAₜGₘCₘUₘAₘGₘAₘAₘAₘUₘAₘGₘCₘAₜAₜGₜUₜUₜAₜAₜAₜAₜUₜAₜAₜGₜGₜCₜUₜ AₜGₜUₜCₜCₜGₜUₜUₜAₜUₜCₜAₘAₘCₘUₘUₘGₘAₘAₘAₘAₘAₘGₘUₘGₘGₘCₘAₘCₘCₘGₘAₘGₘUₘCₘ GₘGₘUₘGₘCₘUₘₛUₘₛUₘₛUₘ (SEQ ID NO: 1). A denotes an adenine nucleobase (or an analogue thereof). C denotes a cytosine nucleobase (or an analogue thereof). G denotes a guanine nucleobase (or an analogue thereof). U denotes an uracil nucleobase (or an analogue thereof). t denotes a 2′-O-methyldithiomethyl (2′-O-MDTM) modified sugar moiety. m denotes a 2′-O-methyl (2′-O-M) modified sugar moiety. N is A, C, G, or U. s denotes a phosphorothioate (ps) internucleotide linkage.

In some embodiments, an oligomeric compound comprising a modified oligonucleotide according to the following formula:

nₛnₛnₛNNNNNNNNNNNNNNNNN

GUUUUAGAgcuagaaauagcAAGUUAAAAUAAGGCUAGUCCGUUAUCaacuugaaaaagu ggcaccgagucggugcuₛuₛuₛu (SEQ ID NO:2). A denotes a 2′-O-methyldithiomethyl-adenosine (or an analogue thereof). C denotes a 2′-O-methyldithiomethyl-cytodine (or an analogue thereof). G denotes a 2′-O-methyldithiomethyl-guanosine (or an analogue thereof). U denotes a 2′-O-methyldithiomethyl-uradine (or an analogue thereof). N is A, C, G, or U. a denotes a 2′-O-methyl-adenosine (or an analogue thereof). c denotes a 2′-O-methyl-cytodine (or an analogue thereof). g denotes a 2′-O-methyl-guanosine (or an analogue thereof). u denotes a 2′-O-methyl-uradine (or an analogue thereof). n is a, c, g, or u. s denotes a phosphorothioate (ps) internucleotide linkage.

Disclosed herein include embodiments of a ribonucleic acid (RNA), such as a single guide ribonucleic acid (sgRNA). In some embodiments, a sgRNA has the following formula:

5′—guide segment—repeat segment—tetraloop—anti-repeat segment—stem loop 1—linker—stem loop 2—stem loop 3—terminator segment −3′.

Nucleotides at positions 1-3 of guide segment can comprise 2′-O-methyl modified sugar moieties. Nucleosides of tetraloop and the four 3′ most nucleotides of the repeat segment, and the four 5′ most nucleotides of the anti-repeat segment can comprise 2′-O-methyl modified sugar moieties. Nucleotides of stem loop 2, stem loop 3, and terminator segment can comprise 2′-O-methyl modified sugar moieties. The reminder nucleotides of the sgRNA can comprise 2′-O-methyldithiomethyl modified sugar moieties. The internucleotide linkages between nucleosides at positions 1-4 of guide segment can comprise phosphorothioate internucleotide linkages. The internucleotide linkages between the last two to four nucleosides of terminator segment can comprise phosphorothioate internucleotide linkages.

In some embodiments, a single guide ribonucleic acid (sgRNA) has the following formula:

5′—guide segment—repeat segment—tetraloop—anti-repeat segment—stem loop 1—linker—stem loop 2—stem loop 3—terminator segment −3′.

Guide segment, repeat segment, anti-repeat segment, stem loop 1, and/or linker can comprise one or more nucleotides each comprising a 2′-O-methyldithiomethyl modified sugar moiety. Guide segment, repeat segment, tetraloop, anti-repeat segment, stem loop 2, stem loop 3, and/or terminator segment can comprise one or more nucleotides each comprising a 2′-O-methyl modified sugar moiety.

In some embodiments, a single guide ribonucleic acid (sgRNA) has the following formula:

5′—guide segment—repeat segment—tetraloop—anti-repeat segment—stem loop 1—linker—stem loop 2—stem loop 3—terminator segment −3′.

Guide segment, repeat segment, anti-repeat segment, stem loop 1, and/or linker can comprise one or more nucleotides each comprising a 2′-O-methyldithiomethyl modified sugar moiety.

In some embodiments, guide segment, repeat segment, tetraloop, anti-repeat segment, stem loop 2, stem loop 3, and/or terminator segment comprises one or more nucleotides, each of the one or more nucleotides comprising a modified sugar moiety. In some embodiments, guide segment, repeat segment, tetraloop, anti-repeat segment, stem loop 2, stem loop 3, and/or terminator segment comprises one or more nucleotides, each of the one or more nucleotides comprising a modification in the backbone of the sgRNA.

In some embodiments, the modified sugar moiety comprises 2′-O-methyl (2′-Me), 2′ fluoro (2′-F), 2′-O-methoxy-ethyl (2′-MOE), a (S)-constrained ethyl (cEt), a locked nucleic acid (LNA), an unlocked nucleic acid (UNA), a bridged nucleic acid, an ethylene-bridged nucleic acid (ENA), or a deoxynucleic acid (DNA). In some embodiments, the modification in the backbone of the sgRNA comprises a peptide nucleic acid (PNA), a phosphorothioate (PS) a phosphorodiamidate morpholino (PMO), a phosphoramidate, or a thiophosphoramidate.

In some embodiments, the internucleotide linkages between two nucleosides of the guide segment comprises a phosphorothioate internucleotide linkage. The internucleotide linkage between nucleosides at positions 1-2 can comprise a phosphorothioate internucleotide linkage. The internucleotide linkages between nucleosides at positions 1-4 can comprise phosphorothioate internucleotide linkages. The internucleotide linkage between two nucleotides of terminator segment can comprise a phosphorothioate internucleotide linkage. The internucleotide linkage between the last two nucleotides of terminator segment can comprise a phosphorothioate internucleotide linkage. The internucleotide linkages between the last four nucleosides of terminator segment can comprise phosphorothioate internucleotide linkages.

In some embodiments, guide segment is complementary to a sequence of a target. The target can be a mammalian target, such as a human target. In some embodiments, guide segment comprises 20 nucleotides or about 20 nucleotides. In some embodiments, nucleotides at one, one or more, or each of positions 4 to 20 of guide segment each comprises a 2′-O-methyldithiomethyl modified sugar moiety. Guide segment can comprise 1 to 17 nucleotides each comprises a 2′-O-methyldithiomethyl modified sugar moiety. In some embodiments, nucleotides at one, one or more, or each of positions 1 to 3 of guide segment each comprises a modified sugar moiety, or a 2′-O-methyl modified sugar moiety. Guide segment can comprise 1 to 3 nucleotides each comprises a modified sugar moiety or a 2′-O-methyl modified sugar moiety.

In some embodiments, repeat segment is 12 nucleotides or about 12 nucleotides in length. In some embodiments, nucleotides at one, one or more, or each of positions 1 to 8 of repeat segment each comprises a 2′-O-methyldithiomethyl modified sugar moiety. Repeat segment can comprise 1 to 8 nucleotides each comprises a 2′-O-methyldithiomethyl modified sugar moiety. In some embodiments, nucleotides at one, one or more, or each of positions 9 to 12 of repeat segment each comprises a 2′-O-methyl modified sugar moiety. Repeat segment can comprise 1 to 4 nucleotides each comprises a 2′-O-methy modified sugar moiety.

In some embodiments, tetraloop is 4 nucleotides or about 4 nucleotides in length. In some embodiments, nucleotides at one, one or more, or each of positions 1 to 4 of tetraloop each comprises a 2′-O-methyl modified sugar moiety. Tetraloop can comprise 1 to 4 nucleotides each comprises a 2′-O-methy modified sugar moiety.

An anti-repeat segment can be 14 nucleotides or about 14 nucleotides in length. In some embodiments, nucleotides at one, one or more, or each of positions 5 to 14 of anti-repeat segment each comprises a 2′-O-methyldithiomethyl modified sugar moiety. Anti-repeat segment can comprise 1 to 10 nucleotides each comprises a 2′-O-methyldithiomethyl modified sugar moiety. In some embodiments, nucleotides at one, one or more, or each of positions 1 to 4 of anti-repeat segment each comprises a 2′-O-methyl modified sugar moiety. Anti-repeat segment can comprise 1 to 4 nucleotides each comprises a 2′-O-methyl modified sugar moiety.

In some embodiments, stem loop 1 is 9 to 13 nucleotides or about 9 to 13 nucleotides in length. In some embodiments, nucleotides at one, one or more, or each of positions 1 to 9, 1 to 11, or 1 to 13, of stem loop 1 each comprises a 2′-O-methyldithiomethyl modified sugar moiety. Stem loop 1 can comprise 9, 11, or 13, nucleotides each comprises a 2′-O-methyldithiomethyl modified sugar moiety.

The linker can be 4 or 5 nucleotides or about 4 or about 5 nucleotides in length. In some embodiments, nucleotides at one, one or more, or each of positions 1 to 4, or 1 to 5, of linker each comprises a 2′-O-methyldithiomethyl modified sugar moiety. Linker can comprise 4 or 5 nucleotides each comprises a 2′-O-methyldithiomethyl modified sugar moiety.

The stem loop 2 can be 12 or 14 nucleotides or about 12 or 14 nucleotides in length. In some embodiments, nucleotides at one, one or more, or each of positions 1 to 12, or 1 to 14, of stem loop 2 each comprises a 2′-O-methyl modified sugar moiety. Stem loop 2 can comprise 1 to 12, or 1 to 14, nucleotides each comprises a 2′-O-methyl modified sugar moiety.

In some embodiments, stem loop 3 is 15 nucleotides or about 15 nucleotides in length. In some embodiments, nucleotides at one, one or more, or each of positions 1 to 15 of stem loop 3 each comprises a 2′-O-methyl modified sugar moiety. Stem loop 3 can comprise 1 to 15 nucleotides each comprises a 2′-O-methyl modified sugar moiety.

In some embodiments, terminator segment is 4 nucleotides or about 4 nucleotides in length. In some embodiments, nucleotides at one, one or more, or each of positions 1 to 4 of terminator segment each comprises a 2′-O-methyl modified sugar moiety. Terminator segment can comprise 1 to 4 nucleotides each comprises a 2′-O-methyl modified sugar moiety.

In some embodiments, the sequence of the sgRNA comprises the sequence of

nnnNNNNNNNNNNNNNNNNN

GUUUUAGAgcuagaaauagcAAGUUAAAAUAAGGCUAGUCCGUUAUCaacuugaaaaagu ggcaccgagucggugcuuuu (SEQ ID NO:3). In some embodiments, the sgRNA comprises a nucleic acid sequence that is at least 90% identical to SEQ ID NO: 3. The sgRNA can comprise a nucleic acid sequence with one or more deletion(s), insertion(s), or substitution(s), relative to SEQ ID NO: 3.

In some embodiments, guide segment comprises the sequence of nucleotides at positions 1 to 20 of SEQ ID NO:3. Repeat segment can comprise the sequence of nucleotides at positions 21 to 32 of SEQ ID NO: 3, and optionally with insertion(s), deletion(s), and/or substitution(s). Tetraloop can comprise the sequence of nucleotides at positions 33 to 36 of SEQ ID NO: 3, and optionally with insertion(s), deletion(s), and/or substitution(s). Anti-repeat segment can comprise the sequence of nucleotides at positions 37 to 50 of SEQ ID NO: 3, and optionally with insertion(s), deletion(s), and/or substitution(s). Stem loop 1 can comprise the sequence of nucleotides at positions 53 to 61, 52 to 62, or 51 to 63, of SEQ ID NO: 3, and optionally with insertion(s), deletion(s), and/or substitution(s). Linker can comprise the sequence of nucleotides at positions 63 to 67, or 64 to 67, of SEQ ID NO: 3, and optionally with insertion(s), deletion(s), and/or substitution(s). Stem loop 2 can comprise the sequence of nucleotides at positions 69 to 80, or 68 to 81, of SEQ ID NO: 3, and optionally with insertion(s), deletion(s), and/or substitution(s). Stem loop 3 can comprise the sequence of nucleotides at positions 82 to 96 of SEQ ID NO: 3, and optionally with insertion(s), deletion(s), and/or substitution(s). Terminator segment can comprise the sequence of nucleotides at positions 97 to 100 of SEQ ID NO: 3, and optionally with insertion(s), deletion(s), and/or substitution(s).

In some embodiments, guide segment and repeat segment are separated by at least one nucleotide. Repeat segment and tetraloop can be separated by at least one nucleotide. Tetraloop and anti-repeat segment can be separated by at least one nucleotide. Anti-repeat segment and stem loop 1 can be separated by at least one nucleotide. Stem loop 1 and linker can be separated by at least one nucleotide. Linker and stem loop 2 can be separated by at least one nucleotide. Stem loop 2 and stem loop 3 can be separated by at least one nucleotide. Stem loop 3 and terminator segment can be separated by at least one nucleotide. In some embodiments, the sgRNA is 100 nucleotides, or about 100 nucleotides, in length.

In some embodiments, the sgRNA comprises a N-Acetylgalactosamine (GalNAc) or a triantennary GalNAc on the 3′-end.

In some embodiments, an adenine nucleobase comprises an adenine nucleobase analogue. A cytosine nucleobase comprises a cytosine nucleobase analogue. A guanine nucleobase can comprise a guanine nucleobase analogue. An uracil nucleobase can comprise an uracil nucleobase analogue. In some embodiments, the adenine nucleobase analogue comprises

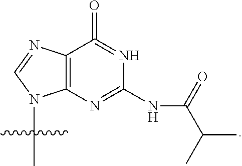

The cytosine nucleobase analogue can comprise

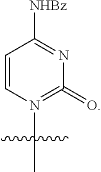

The guanine nucleobase analogue can comprise

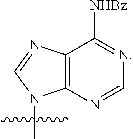

In some embodiments, a nucleobase analogue comprises 2-aminopurine, thymine, 2,6-diaminopurine, 2-pyrimidinone, 5-methylcytosine, 5-hydroxymethylcytosine, hypoxanthine, xanthine, 7-methylguanine, or 5,6-dihydrouracil.

Disclosed herein include embodiments of a composition. The composition can comprise an oligomeric compound or a sgRNA of the present disclosure.

Disclosed herein include embodiments of a compositions. In some embodiments, a composition comprises the oligomeric compound or a sgRNA of the present disclosure (e.g., with the disulfide bonds of the 2′-O-methyldithiomethyl groups of modified sugar moieties of the modified oligonucleotide of the oligomeric compound, or sgRNA, reduced, forming 2′-O-methanethiol groups, which are then converted to 2′-OH) in complex with an RNA-guided endonuclease. The RNA-guided endonuclease can be a small Cas nuclease or a small RNA-guided endonuclease. The RNA-guided endonuclease can be a Cas9, a Cas12, aCas13, or a variant thereof. The RNA-guided endonuclease can be a Streptococcus pyogenes Cas9 (SpyCas9) or a Staphylococcus aureus Cas9 (SaCas9). The RNA-guided endonuclease can be a variant of Cas9. The variant of Cas9 can be a small Cas9, a dead Cas9 (dCas9), or a Cas9 nickase, The RNA-guided endonuclease can be a Campylobacter jejuni Cas9 (CjCas9).

Disclosed herein include methods of generating an oligomeric compound or a sgRNA of the present disclosure. In some embodiments, a method of generating an oligomeric compound comprises chemically synthesizing a precursor nucleic acid comprising the sequence of the modified oligonucleotide. A nucleotide with a 2′-O-methyldithiomethyl modified sugar moiety in the oligomeric compound is synthesized using

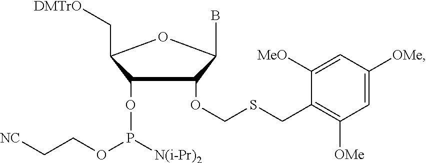

where B is the nucleobase of the nucleotide with the 2′-O-methyldithiomethyl modified sugar moiety. The method can comprise reacting the precursor nucleic acid with dimethyl(methylthio)-sulfonium tetrafluoroborate (DMTSF) to generate the oligomeric compound.

In some embodiments, a method of generating a sgRNA comprises chemically synthesizing a precursor nucleic acid comprising the sequence of the sgRNA. A nucleotide with a 2′-O-methyldithiomethyl modified sugar moiety in the sgRNA is synthesized using

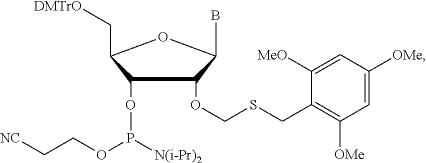

where B is the nucleobase of the nucleotide with the 2′-O-methyldithiomethyl modified sugar moiety. The method can comprise reacting the precursor nucleic acid with dimethyl(methylthio)-sulfonium tetrafluoroborate (DMTSF) to generate the sgRNA.

In some embodiments, the precursor nucleic acid is attached to a solid support. The method can comprise cleaving the precursor nucleic acid attached to the solid support to release the precursor nucleic acid from the solid support. In some embodiments, the method can comprise purifying the sgRNA generated, optionally wherein the sgRNA is purified using high-performance liquid chromatography (HPLC).

Disclosed herein include methods of treating a subject in need thereof. In some embodiments, a method of treating a subject in need thereof comprises administering an oligomeric compound, a sgRNA, or a composition of the present disclosure to a subject in need thereof. The disulfide bond of a 2′-O-methyldithiomethyl group of a modified sugar moiety of the administered modified oligonucleotide, or sgRNA, can be reduced to a 2′-O-methanethiol group in a cell of the subject. The 2′-O-methanethiol group can be converted to 2′-OH. An RNA-guided endonuclease binds the modified oligonucleotide, or sgRNA.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 shows exemplary modifications of oligonucleotides.

FIG. 2A-B are the HPLC/MS profiles of the oligonucleotides before DMTSF treatment with the dimethoxytrityl (DMT) protecting group off (FIG. 2A) or on (FIG. 2B).

FIG. 3A-3G are the LCMS HPCL profiles of the oligonucleotides after the DMTSF treatment trials of Table 1 (FIG. 3A: trial 2.1, 2.2 with water and 2.3 with EtOH; FIG. 3B: trial 3.1, 3.2 and 3.3; FIG. 3C: trial 4.1 and 4.2; FIG. 3D: trial 5.1 with 4 h reaction time; FIG. 3E: trial 5.1 with 24 h reaction time; FIG. 3F: trial 5.2 with 4 h reaction time; and FIG. 3G: trial 5.2 with 24 h reaction time).

FIG. 4 is the LCMS HPCL profile after a DMTSF treatment.

FIG. 5A is a HPLC purification profile of exemplary oligonucleotide mT2-RNA1.

FIG. 5B shows the fraction analysis (RP-LCMS) profile of exemplary oligonucleotide mT2-RNA1 with HFIP/TEA/MeOH gradient.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

An endonuclease can cleave internally of an RNA. A 5′-exonuclease can cleave on the 5′-end of an endonuclease. A 3′-exonuclease can cleave on the 3′-end of an endonuclease. Endonucleases and exonucleases are ubiquitously present in both plasma and tissues. Endonucleases and exonucleases cannot cleave RNA (e.g., gRNA or sgRNA) of the present disclosure.

Disclosed herein include embodiments of an RNA or an oligomeric compound comprising a modified oligonucleotide according to the following formula:

NₘₛNₘₛNₘₛNₜNₜNₜNₜNₜNₜNₜNₜNₜNₜNₜNₜNₜNₜNₜNₜNₜ

GₜUₜUₜUₜUₜAₜG_{T}A_{T}GₘCₘUₘAₘGₘAₘAₘAₘUₘAₘGₘCₘA_{T}A_{T}G_{T}U_{T}U_{T}A_{T}A_{T}A_{T}A_{T}U_{T}A_{T}A_{T}G_{T} G_{T}C_{T}U_{T}A_{T}G_{T}U_{T}C_{T}C_{T}G_{T}U_{T}U_{T}A_{T}U_{T}C_{T}AₘAₘCₘUₘUₘGₘAₘAₘAₘAₘAₘGₘUₘGₘGₘCₘAₘCₘCₘGm AₘGₘUₘCₘGₘGₘUₘGₘCₘUₘₛUₘₛUₘₛUₘ (SEQ ID NO: 1). A denotes an adenine nucleobase (or an analogue thereof). C denotes a cytosine nucleobase (or an analogue thereof). G denotes a guanine nucleobase (or an analogue thereof). U denotes an uracil nucleobase (or an analogue thereof). t denotes a 2′-O-methyldithiomethyl (2′-O-MDTM) modified sugar moiety. m denotes a 2′-O-methyl (2′-O-M) modified sugar moiety. N is A, C, G, or U. s denotes a phosphorothioate (ps) internucleotide linkage.

Disclosed herein include embodiments of an RNA or an oligomeric compound comprising a modified oligonucleotide according to the following formula:

nₛnₛnₛNNNNNNNNNNNNNNNNN

GUUUUAGAgcuagaaauagcAAGUUAAAAUAAGGCUAGUCCGUUAUCaacuugaaaaagu ggcaccgagucggugcuₛuₛuₛu (SEQ ID NO: 2). A denotes a 2′-O-methyldithiomethyl-adenosine (or an analogue thereof). C denotes a 2′-O-methyldithiomethyl-cytodine (or an analogue thereof). G denotes a 2′-O-methyldithiomethyl-guanosine (or an analogue thereof). U denotes a 2′-O-methyldithiomethyl-uradine (or an analogue thereof). N is A, C, G, or U. a denotes a 2′-O-methyl-adenosine (or an analogue thereof). c denotes a 2′-O-methyl-cytodine (or an analogue thereof). g denotes a 2′-O-methyl-guanosine (or an analogue thereof). u denotes a 2′-O-methyl-uradine (or an analogue thereof). n is a, c, g, or u. s denotes a phosphorothioate (ps) internucleotide linkage.

Disclosed herein include embodiments of a single guide ribonucleic acid (sgRNA) according to the following formula:

5′—guide segment—repeat segment—tetraloop—anti-repeat segment—stem loop 1—linker—stem loop 2—stem loop 3—terminator segment −3′.

Nucleotides at positions 1-3 of guide segment can comprise 2′-O-methyl modified sugar moieties. Nucleosides of tetraloop and the four 3′ most nucleotides of the repeat segment, and the four 5′ most nucleotides of the anti-repeat segment can comprise 2′-O-methyl modified sugar moieties. Nucleotides of stem loop 2, stem loop 3, and terminator segment can comprise 2′-O-methyl modified sugar moieties. The reminder nucleotides of the sgRNA can comprise 2′-O-methyldithiomethyl modified sugar moieties. The internucleotide linkages between nucleosides at positions 1-4 of guide segment can comprise phosphorothioate internucleotide linkages. The internucleotide linkages between the last two to four nucleosides of terminator segment can comprise phosphorothioate internucleotide linkages.

Disclosed herein include embodiments of a single guide ribonucleic acid (sgRNA) according to the following formula:

5′—guide segment—repeat segment—tetraloop—anti-repeat segment—stem loop 1—linker—stem loop 2—stem loop 3—terminator segment −3′.

Guide segment, repeat segment, anti-repeat segment, stem loop 1, and/or linker can comprise one or more nucleotides each comprising a 2′-O-methyldithiomethyl modified sugar moiety. Guide segment, repeat segment, tetraloop, anti-repeat segment, stem loop 2, stem loop 3, and/or terminator segment can comprise one or more nucleotides each comprising a 2′-O-methyl modified sugar moiety.

Disclosed herein include embodiments of a single guide ribonucleic acid (sgRNA) according to the following formula:

5′—guide segment—repeat segment—tetraloop—anti-repeat segment—stem loop 1—linker—stem loop 2—stem loop 3—terminator segment −3′.

Guide segment, repeat segment, anti-repeat segment, stem loop 1, and/or linker can comprise one or more nucleotides each comprising a 2′-O-methyldithiomethyl modified sugar moiety.

Disclosed herein include embodiments of a composition comprising an oligomeric compound or a sgRNA of the present disclosure. Disclosed herein include embodiments of a compositions comprising an oligomeric compound or a sgRNA of the present disclosure in complex with an RNA-guided endonuclease (e.g., with the disulfide bonds of 2′-O-methyldithiomethyl groups of modified sugar moieties of the modified oligonucleotide of oligomeric compound, or sgRNA, reduced, forming 2′-O-methanethiol groups in a cell of the subject, which is then converted (e.g., spontaneously converted) to 2′-OH in the cell).

Disclosed herein include methods of generating an oligomeric compound of the present disclosure comprising chemically synthesizing a precursor nucleic acid comprising the sequence of the modified oligonucleotide. A nucleotide with a 2′-O-methyldithiomethyl modified sugar moiety in the oligomeric compound is synthesized using

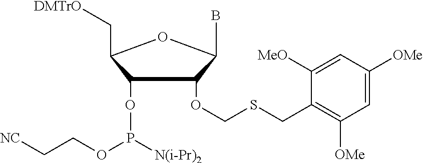

where B is the nucleobase of the nucleotide with the 2′-O-methyldithiomethyl modified sugar moiety. The method can comprise reacting the precursor nucleic acid with dimethyl(methylthio)-sulfonium tetrafluoroborate (DMTSF) to generate the oligomeric compound.

Disclosed herein include embodiments of a method of generating a sgRNA of the present disclosure comprising chemically synthesizing a precursor nucleic acid comprising the sequence of the sgRNA. A nucleotide with a 2′-O-methyldithiomethyl modified sugar moiety in the sgRNA is synthesized using

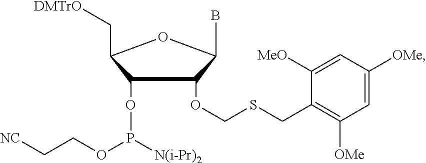

where B is the nucleobase of the nucleotide with the 2′-O-methyldithiomethyl modified sugar moiety. The method can comprise reacting the precursor nucleic acid with dimethyl(methylthio)-sulfonium tetrafluoroborate (DMTSF) to generate the sgRNA.

Disclosed herein include embodiments of a method of treating a subject in need thereof comprising administering an oligomeric compound, a sgRNA, or a composition of the present disclosure.

### Definitions

As used herein, the term “about” means plus or minus 5% of the provided value.

As used herein, the term “moderate length RNA (RNA)” refers an RNA molecule with a length of about 30 to about 160 nucleotides. An RNA can be or be about 30, 35, 40, 45, 50, 55, 60, 65, 70, 75, 80, 85, 90, 91, 92, 93, 94, 95, 96, 97, 98, 99, 100, 101, 102, 103, 104, 105, 106, 107, 108, 109, 110, 115, 120, 125, 130, or a number or a range between any two of these values, nucleotides in length. For example, an RNA can be a RNA (e.g., gRNA) of 100 nucleotides in length.

As used herein, the term “RNA-guided endonuclease” refers to a polypeptide capable of binding a RNA (e.g., a gRNA) to form a complex targeted to a specific DNA sequence (e.g., in a target DNA). A non-limiting example of RNA-guided endonuclease is a Cas polypeptide (e.g., a Cas endonuclease, such as a Cas9 endonuclease). In some embodiments, the RNA-guided endonuclease as described herein is targeted to a specific DNA sequence in a target DNA by an RNA molecule to which it is bound. The RNA molecule can include a sequence that is complementary to and capable of hybridizing with a target sequence within the target DNA, thus allowing for targeting of the bound polypeptide to a specific location within the target DNA.

As used herein, the term “guide RNA” or “gRNA” refers to a site-specific targeting RNA that can bind an RNA-guided endonuclease to form a complex, and direct the activities of the bound RNA-guided endonuclease (such as a Cas endonuclease) to a specific target sequence within a target nucleic acid. The guide RNA can include one or more RNA molecules.

As used herein, a “secondary structure” of a nucleic acid molecule (e.g., an RNA fragment, or a gRNA) refers to the base pairing interactions within the nucleic acid molecule.

As used herein, the term “target DNA” refers to a DNA that includes a “target site” or “target sequence.” The term “target sequence” is used herein to refer to a nucleic acid sequence present in a target DNA to which a DNA-targeting sequence or segment (also referred to herein as a “spacer”) of a gRNA can hybridize, provided sufficient conditions for hybridization exist. For example, the target sequence 5′-GAGCATATC-3′ within a target DNA is targeted by (or is capable of hybridizing with, or is complementary to) the RNA sequence 5′-GAUAUGCUC-3′. Hybridization between the DNA-targeting sequence or segment of a gRNA and the target sequence can, for example, be based on Watson-Crick base pairing rules, which enables programmability in the DNA-targeting sequence or segment. The DNA-targeting sequence or segment of a gRNA can be designed, for instance, to hybridize with any target sequence.

As used herein, the term “Cas endonuclease” or “Cas nuclease” refers to an RNA-guided DNA endonuclease associated with the CRISPR adaptive immunity system.

Unless otherwise indicated “nuclease” and “endonuclease” are used interchangeably herein to refer to an enzyme which possesses endonucleolytic catalytic activity for polynucleotide cleavage.

As used herein, the term “cleavage” refers to the breakage of the covalent backbone of a DNA molecule. The cleavage can be a single-stranded cleavage or a double-stranded cleavage. For example, a double-stranded cleavage can occur as a result of two distinct single-stranded cleavage events.

As used herein, the term “domain” refers to a segment of a protein or a nucleic acid. Unless otherwise indicated, a domain need not have any specific functional property.

As used herein, the term “splint oligonucleotide” refers to an oligonucleotide that, when hybridized to other polynucleotides (e.g., RNA fragments), acts as a “splint” to position the ends of the polynucleotides next to one another so that they can be ligated together. The splint oligonucleotide can be any oligomer that can hybridize with the polynucleotides through Watson-Crick base-pairing interactions. The splint oligonucleotide can be DNA, RNA, non-natural, or artificial nucleic acids (e.g., peptide nucleic acids). The splint oligonucleotide can include a nucleotide sequence that is partially complimentary to nucleotide sequences from two or more different oligonucleotides. In general, an RNA ligase, a DNA ligase, or another variety of ligase can be used to ligate two nucleotide sequences together. In some embodiments, the splint oligonucleotide is a DNA oligonucleotide that can be digested by DNase. In some embodiments, the splint oligonucleotide is an oligonucleotide susceptible to digestion by DNase. The splint oligonucleotide can include one or more modifications on one or more sugar moieties and/or one or more bases. In some embodiments, the splint oligonucleotide comprises one or more modified and/or non-natural bases.

As used herein, the “spacer” or “variable region” of a gRNA includes a nucleotide sequence that is complementary to a specific sequence within a target DNA (the complementary strand of the target DNA). In some aspects, the spacer confers target specificity to the gRNA combined with an RNA-guided endonuclease, enabling the RNA-guided endonuclease to cleave at the target site targeted by the spacer in the target DNA. As used herein, the term “spacer” is used interchangeably with the term “spacer sequence.”

As used herein, the term “invariable region” of a gRNA refers to the nucleotide sequence of the gRNA that associates with the RNA-guided endonuclease. In some embodiments, the gRNA comprises a crRNA and a transactivating crRNA (tracrRNA), wherein the crRNA and tracrRNA hybridize to each other to form a duplex. In some embodiments, the crRNA comprises 5′ to 3′: a spacer sequence and minimum CRISPR repeat sequence (also referred to as a “crRNA repeat sequence” herein); and the tracrRNA comprises a minimum tracrRNA sequence complementary to the minimum CRISPR repeat sequence (also referred to as a “tracrRNA anti-repeat sequence” herein) and a 3′ tracrRNA sequence. In some embodiments, the invariable region of the gRNA refers to the portion of the crRNA that is the minimum CRISPR repeat sequence and the tracrRNA.

The terms “polynucleotide” and “nucleic acid” are used interchangeably herein and refer to a polymeric form of nucleotides of any length, either ribonucleotides or deoxyribonucleotides. A polynucleotide can be single-, double-, or multi-stranded DNA or RNA, genomic DNA, cDNA, DNA-RNA hybrids/triple helices, or a polymer including purine and pyrimidine bases or other natural, chemically or biochemically modified, non-natural, or derivatized nucleotide bases.

As used herein, the term “binding” refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (K_{d}), for example a K_{d} of, or a K_{d} less than, 10⁻⁶ M, 10⁻⁷ M, 10⁻¹ M, 10⁻⁹ M, 10⁻¹⁰ M, 10⁻¹¹ M, 10⁻¹² M, 10⁻¹³ M, 10⁻¹⁴ M, 10⁻¹⁵ M, or a number or a range between any two of these values. K_{d} can be dependent on environmental conditions, e.g., pH and temperature. “Affinity” refers to the strength of binding, and increased binding affinity is correlated with a lower K_{d}.

As used herein, the term “hybridizing” or “hybridize” refers to the pairing of substantially complementary or complementary nucleic acid sequences within two different molecules. Pairing can be achieved by any process in which a nucleic acid sequence joins with a substantially or fully complementary sequence through base pairing to form a hybridization complex. “Hybridizing” or “hybridize” can comprise denaturing the molecules to disrupt the intramolecular structure(s) (e.g., secondary structure(s)) in the molecule. In some embodiments, denaturing the molecules comprises heating a solution comprising the molecules to a temperature sufficient to disrupt the intramolecular structures of the molecules. In some instances, denaturing the molecules comprises adjusting the pH of a solution comprising the molecules to a pH sufficient to disrupt the intramolecular structures of the molecules. For purposes of hybridization, two nucleic acid sequences or segments of sequences are “substantially complementary” if at least 80% of their individual bases are complementary to one another. In some embodiments, a splint oligonucleotide sequence is not more than about 50% identical to one of the two polynucleotides (e.g., RNA fragments) to which it is designed to be complementary. The complementary portion of each sequence can be referred to herein as a ‘segment’, and the segments are substantially complementary if they have 80% or greater identity.

### Guide Ribonucleic Acid

The present disclosure provides RNA (e.g., gRNA or sgRNA), referred to herein as prodrug RNA, comprising one or more modification such as one or more 2′-O-methyldithiomethyl modified sugar moieties (referred to herein as transient protecting groups). The RNA can have enhanced nuclease stability. The RNA can overcome nuclease degradation. The RNA can be resistant to exonuclease and endonuclease cleavage. The RNA can be conditionally activatable. The RNA can have increased chemical stability. The RNA can have decreased immunogenicity. The RNA (e.g., in complex with an RNA-guided endonuclease) can have reduced off-target effects. The RNA (e.g., in complex with an RNA-guided endonuclease) can have enhanced efficacy. The RNA can be, for example, a, TA1 gRNA, B2M gRNA, CD70 gRNA, Ctx.2 gRNA, SPY101 gRNA or mTF_T2 sgRNA.

Disclosed herein include embodiments of an RNA or oligomeric compound (e.g. a single guide RNA). In some embodiments, an oligomeric compound comprises a modified oligonucleotide (referred to herein as a prodrug RNA) according to the following formula:

NₘₛNₘₛNₘₛNₜNₜNₜNₜNₜNₜNₜNₜNₜNₜNₜNₜNₜNₜNₜNₜNₜ

GₜUₜUₜUₜUₜAₜG_{T}A_{T}GₘCₘUₘAₘGₘAₘAₘAₘUₘAₘGₘCₘA_{T}A_{T}G_{T}U_{T}U_{T}A_{T}A_{T}A_{T}A_{T}U_{T}A_{T}A_{T}G_{T} G_{T}C_{T}U_{T}A_{T}G_{T}U_{T}C_{T}C_{T}G_{T}U_{T}U_{T}A_{T}U_{T}C_{T}AₘAₘCₘUₘUₘGₘAₘAₘAₘAₘAₘGₘUₘGₘGₘCₘAₘCₘCₘGm AₘGₘUₘCₘGₘGₘUₘGₘCₘUₘₛUₘₛUₘₛUₘ (SEQ ID NO: 1). A denotes an adenine nucleobase (or an analogue thereof). C denotes a cytosine nucleobase (or an analogue thereof). G denotes a guanine nucleobase (or an analogue thereof). U denotes an uracil nucleobase (or an analogue thereof). t denotes a 2′-O-methyldithiomethyl (2′-O-MDTM) modified sugar moiety. m denotes a 2′-O-methyl (2′-O-M) modified sugar moiety. N is A, C, G, or U. s denotes a phosphorothioate (ps) internucleotide linkage.

In some embodiments, an RNA or oligomeric compound (e.g., a single guide RNA) comprising a modified oligonucleotide (referred to herein as a prodrug RNA) according to the following formula:

nₛnₛnₛNNNNNNNNNNNNNNNNN

GUUUUAGAgcuagaaauagcAAGUUAAAAUAAGGCUAGUCCGUUAUCaacuugaaaaagu ggcaccgagucggugcuₛuₛuₛu (SEQ ID NO: 2). A denotes a 2′-O-methyldithiomethyl-adenosine (or an analogue thereof). C denotes a 2′-O-methyldithiomethyl-cytodine (or an analogue thereof). G denotes a 2′-O-methyldithiomethyl-guanosine (or an analogue thereof). U denotes a 2′-O-methyldithiomethyl-uradine (or an analogue thereof). N is A, C, G, or U. a denotes a 2′-O-methyl-adenosine (or an analogue thereof). c denotes a 2′-O-methyl-cytodine (or an analogue thereof). g denotes a 2′-O-methyl-guanosine (or an analogue thereof). u denotes a 2′-O-methyl-uradine (or an analogue thereof). n is a, c, g, or u. s denotes a phosphorothioate (ps) internucleotide linkage.

Disclosed herein include embodiments of a ribonucleic acid (RNA), such as a single guide ribonucleic acid (sgRNA) which is referred to herein as a prodrug RNA. In some embodiments, a sgRNA has the following formula:

5′—guide segment—repeat segment—tetraloop—anti-repeat segment—stem loop 1—linker—stem loop 2—stem loop 3—terminator segment −3′.

The variable region of the sgRNA can comprise guide segment. The invariable region of the sgRNA can comprise repeat segment, tetraloop, anti-repeat segment, stem loop 1, linker, stem loop 2, stem loop 3, and terminator segment. One or more nucleotides (e.g., the nucleotides at positions 1-3) of guide segment can comprise modified sugar moieties, such as 2′-O-methyl modified sugar moieties. One or more nucleotides (e.g., all nucleosides) of tetraloop can comprise modified sugar moieties, such as 2′-O-methyl modified sugar moieties. One or more nucleotides (e.g., the four 3′ most nucleotides) of the repeat segment can comprise modified sugar moieties, such as 2′-O-methyl modified sugar moieties. One or more nucleotides (e.g., the four 5′ most nucleotides) of the anti-repeat segment can comprise modified sugar moieties, such as 2′-O-methyl modified sugar moieties. One or more nucleotides (e.g., all nucleotides) of stem loop 2 can comprise modified sugar moieties, such as 2′-O-methyl modified sugar moieties. One or more nucleotides (e.g., all nucleotides) of stem loop 3 can comprise modified sugar moieties, such as 2′-O-methyl modified sugar moieties. One or more nucleotides (e.g., all nucleotides) of terminator segment can comprise modified sugar moieties, such as 2′-O-methyl modified sugar moieties. One or more reminder nucleotides (e.g., all reminder nucleotides) of the sgRNA can comprise 2′-O-methyldithiomethyl modified sugar moieties (referred to herein as transient protecting groups). A nucleotide comprising a 2′-O-methyldithiomethyl modified sugar moiety can have the structure shown below:

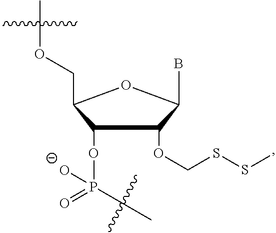

where B is the nucleobase of the nucleotide. A sgRNA comprising one or more 2′-O-methyldithiomethyl modified sugar moieties is referred to herein as prodrug RNA. The internucleotide linkages between two or more nucleotides (e.g., nucleosides at positions 1-4) of guide segment can comprise phosphorothioate internucleotide linkages. The internucleotide linkages between two or more nucleotides (e.g., the last two, three, or four nucleosides) of terminator segment can comprise phosphorothioate internucleotide linkages.

In some embodiments, a single guide ribonucleic acid has the following formula:

5′—guide segment—repeat segment—tetraloop—anti-repeat segment—stem loop 1—linker—stem loop 2—stem loop 3—terminator segment −3′.

One or more nucleotides of guide segment can each comprise a 2′-O-methyldithiomethyl modified sugar moiety. A 2′-O-methyldithiomethyl modified sugar moiety is referred to herein as a transient protecting group. Alternatively or additionally, one or more nucleotides of repeat segment can each comprise a 2′-O-methyldithiomethyl modified sugar moiety. Alternatively or additionally, one or more nucleotides of anti-repeat segment can each comprise a 2′-O-methyldithiomethyl modified sugar moiety. Alternatively or additionally, one or more nucleotides of stem loop 1 can each comprise a 2′-O-methyldithiomethyl modified sugar moiety. Alternatively or additionally, one or more nucleotides of linker can each comprise a 2′-O-methyldithiomethyl modified sugar moiety. A sgRNA comprising one or more 2′-O-methyldithiomethyl modified sugar moieties is referred to herein as prodrug RNA.

One or more nucleotides of guide segment can each comprise a modified sugar moiety, such as a 2′-O-methyl modified sugar moiety. Alternatively or additionally, one or more nucleotides of repeat segment can each comprise a modified sugar moiety, such as a 2′-O-methyl modified sugar moiety. Alternatively or additionally, one or more nucleotides of tetraloop can each comprise a modified sugar moiety, such as a 2′-O-methyl modified sugar moiety. Alternatively or additionally, one or more nucleotides of anti-repeat segment can each comprise a modified sugar moiety, such as a 2′-O-methyl modified sugar moiety. Alternatively or additionally, one or more nucleotides of stem loop 2 can each comprise a modified sugar moiety, such as a 2′-O-methyl modified sugar moiety. Alternatively or additionally, one or more nucleotides of stem loop 3 can each comprise a modified sugar moiety, such as a 2′-O-methyl modified sugar moiety. Alternatively or additionally, one or more nucleotides of terminator segment can each comprise a modified sugar moiety, such as a 2′-O-methyl modified sugar moiety.

In some embodiments, a ribonucleic acid, such as a single guide ribonucleic acid, comprises guide segment, repeat segment, tetraloop, anti-repeat segment, stem loop 1, linker, stem loop 2, stem loop 3, and/or terminator segment −3′. In some embodiments, a sgRNA has the following formula:

5′—guide segment—repeat segment—tetraloop—anti-repeat segment—stem loop 1—linker—stem loop 2—stem loop 3—terminator segment −3′.

In some embodiments, a ribonucleic acid does not include a terminator segment.

In some embodiments, guide segment comprises one or more nucleotides each comprising a 2′-O-methyldithiomethyl modified sugar moiety. Alternatively or additionally, repeat segment can comprise one or more nucleotides each comprising a 2′-O-methyldithiomethyl modified sugar moiety. Alternatively or additionally, anti-repeat segment can comprise one or more nucleotides each comprising a 2′-O-methyldithiomethyl modified sugar moiety. Alternatively or additionally, stem loop 1 can comprise one or more nucleotides each comprising a 2′-O-methyldithiomethyl modified sugar moiety. Alternatively or additionally, linker can comprise one or more nucleotides each comprising a 2′-O-methyldithiomethyl modified sugar moiety. A sgRNA comprising one or more 2′-O-methyldithiomethyl modified sugar moieties is referred to herein as prodrug RNA. In some embodiments, tetraloop, stem loop 2, stem loop 3, and/or terminator segment comprises one or more nucleotides each comprising a modified sugar moiety, such as a 2′-O-methyl modified sugar moiety.

In some embodiments, a ribonucleic acid can be a CRISPR RNA (crRNA) according to the following formula:

5′—guide segment—repeat segment −3′.

In some embodiments, a ribonucleic acid can be a trans-activating crRNA (tracrRNA) according to the following formula:

5′—anti-repeat segment—stem loop 1—linker—stem loop 2—stem loop 3—terminator segment −3′.

In some embodiments, a ribonucleic acid does not include a terminator segment. In some embodiments, guide segment comprises one or more nucleotides each comprising a 2′-O-methyldithiomethyl modified sugar moiety. Alternatively or additionally, repeat segment can comprise one or more nucleotides each comprising a 2′-O-methyldithiomethyl modified sugar moiety. Alternatively or additionally, anti-repeat segment can comprise one or more nucleotides each comprising a 2′-O-methyldithiomethyl modified sugar moiety. Alternatively or additionally, stem loop 1 can comprise one or more nucleotides each comprising a 2′-O-methyldithiomethyl modified sugar moiety. Alternatively or additionally, linker can comprise one or more nucleotides each comprising a 2′-O-methyldithiomethyl modified sugar moiety. A gRNA comprising one or more 2′-O-methyldithiomethyl modified sugar moieties is referred to herein as prodrug RNA. In some embodiments, tetraloop, stem loop 2, stem loop 3, and/or terminator segment comprises one or more nucleotides each comprising a modified sugar moiety, such as a 2′-O-methyl modified sugar moiety.

### Backbone Modifications

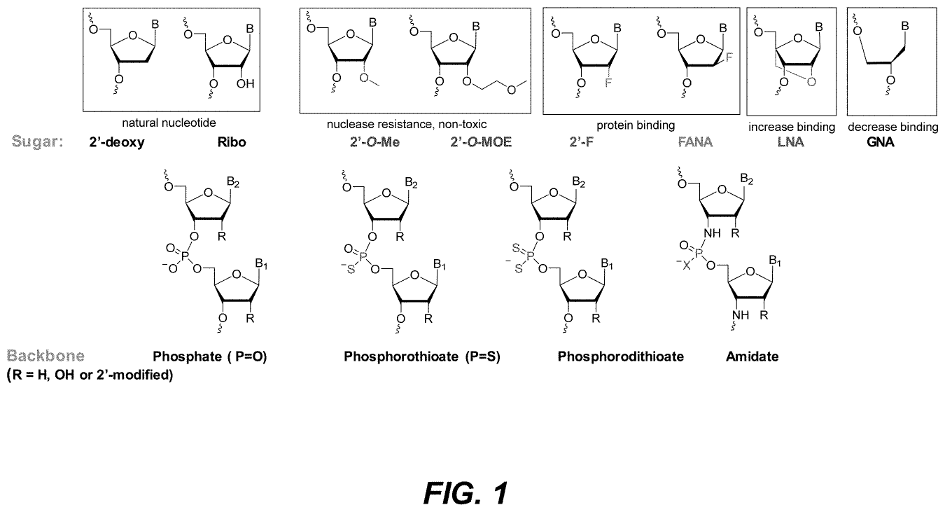

FIG. 1 shows exemplary backbone modifications of oligonucleotides.

In some embodiments, guide segment comprises one or more nucleotides each comprising a modification in the backbone of the RNA, such as a 2′-O-methyl modified sugar moiety. Alternatively or additionally, repeat segment can comprise one or more nucleotides each comprising a modification in the backbone of the RNA, such as a 2′-O-methyl modified sugar moiety. Alternatively or additionally, tetraloop can comprise one or more nucleotides each comprising a modification in the backbone of the RNA, such as a 2′-O-methyl modified sugar moiety. Alternatively or additionally, anti-repeat segment can comprise one or more nucleotides each comprising a modification in the backbone of the RNA, such as a 2′-O-methyl modified sugar moiety. Alternatively or additionally, stem loop 2 can comprise one or more nucleotides each comprising a modification in the backbone of the RNA, such as a 2′-O-methyl modified sugar moiety. Alternatively or additionally, stem loop 3 can comprise one or more nucleotides each comprising a modification in the backbone of the RNA, such as a 2′-O-methyl modified sugar moiety. Alternatively or additionally, terminator segment can comprise one or more nucleotides each comprising a modification in the backbone of the RNA, such as a 2′-O-methyl modified sugar moiety. In some embodiments, stem loop 1 and/or linker comprises one or more nucleotides each comprising a modification in the backbone of the RNA, such as a 2′-O-methyl modified sugar moiety.

In some embodiments, a modified sugar moiety comprises 2′-O-methyl (2′-Me), 2′ fluorine (2′-F), 2′-O-methoxy-ethyl (2′-MOE), a (S)-constrained ethyl (cEt), a locked nucleic acid (LNA), an unlocked nucleic acid (UNA), a bridged nucleic acid, an ethylene-bridged nucleic acid (ENA), or a deoxynucleic acid (DNA). In some embodiments, a modification in the backbone of the RNA comprises a peptide nucleic acid (PNA), a phosphorothioate (PS) a phosphorodiamidate morpholino (PMO), a phosphoramidate, or a thiophosphoramidate.

### Guide

In some embodiments, guide segment is complementary to a sequence of a target. The target can be a mammalian target, such as a human target. In some embodiments, guide segment is, is about, is at least, is at least about, is at most, or is at most about, 15, 16, 17, 18, 19, 20, 21, 22, 23, 24, 25, or a number or a range between any two of these values, in length. For example, guide segment is 20 nucleotides or about 20 nucleotides in length. In some embodiments, guide segment comprises the sequence of G_{S}A_{S}G_{S}AACGCACCACUUUACGA (SEQ ID NO: 4), in which s is a phosphorothioate internucleotide linkage.

In some embodiments, any nucleotide of guide segment can comprise 2′-O-methyldithiomethyl (or a 2′-O-methyldithioalkyl) modified sugar moiety. For example, nucleotides at positions 4, 5, 6, 7, 8 9, 10, 11, 12, 13, 14, 15, 16, 17, 18, 19, and/or 20, of guide segment each comprises a 2′-O-methyldithiomethyl (or a 2′-O-methyldithioalkyl) modified sugar moiety. The nucleotides at positions 1, 2, 3, 4, and/or 5 of guide segment can each comprise a 2′-O-alkyl modified sugar moiety. Guide segment can comprise, comprise about, comprise at least, comprise at least about, comprise at most, or comprise at most about, 0, 1, 2, 3, 4, 5, 6, 7, 8, 9, 10, 11, 12, 13, 14, 15, 16, 17, 18, 19, 20, or a number or a range between any two of these values, nucleotides each comprises a 2′-O-methyldithiomethyl (or a 2′-O-methyldithioalkyl) modified sugar moiety. For example, 17 nucleotides of guide segment each comprises a 2′-O-methyldithiomethyl modified sugar moiety.

In some embodiments, any nucleotide of guide segment can comprise a modified sugar moiety such as a 2′-O-methyl or a 2′-O-alkyl modified sugar moiety. For example, nucleotides at position 1, position 2, and/or position 3 of guide segment each comprises a modified sugar moiety, such as a 2′-O-methyl (or a 2′-O-alkyl) modified sugar moiety. For example, guide segment comprises, comprises about, comprises at least, comprises at least about, comprises at most, or comprises at most about, 0, 1, 2, 3, 4, 5, 6, 7, 8, 9, 10, 11, 12, 13, 14, 15, 16, 17, 18, 19, 20, or a number or a range between any two of these values, nucleotides each comprising a modified sugar moiety, such as a 2′-O-methyl (or a 2′-O-alkyl) modified sugar moiety. For example, guide segment can comprise 3 nucleotides each comprising a modified sugar moiety, such as a 2′-O-methyl (or a 2′-O-alkyl) modified sugar moiety.

The alkyl group of a 2′-O-methyldithioalkyl or a 2′-O-alkyl modified sugar moiety can be methyl, ethyl, propyl, butyl, pentyl, hexyl, heptyl, octyl, nonyl, decyl, undecyl, or dodecyl. The alkyl group of a 2′-O-methyldithioalkyl modified sugar moiety can comprise a carbon chain. The carbon chain can comprise 1, 2, 3, 4, 5, 6, 7, 8, 9, 10, or more, carbon atoms. The carbon chain can be 1, 2, 3, 4, 5, 6, 7, 8, 9, 10, or more, carbon atoms in length. The carbon chain can be substituted. The carbon chain can be unsubstituted. The carbon chain can be saturated. The carbon chain can be unsaturated.

### Repeat

A repeat segment can be, can be about, can be at least, can be at least about, can be at most, or can be at most about, 7, 8, 9, 10, 11, 12, 13, 14, 15, 16, 17, 18, 19, 20, 21, 22, 23, 24, 25, 26, 27, or a number or a range between any two of these values, in length. For example, repeat segment is 12 nucleotides or about 12 nucleotides in length. In some embodiments, nucleotides at position 1, 2, 3, 4, 5, 6, 7, 8, 9, 10, 11, 12, 13, 14, 15, 16, 17, 18, 19, 20, 21, 22, 23, 24, 25, 26, and/or 27, of the repeat segment each comprises a 2′-O-methyldithiomethyl (or 2′-O-methyldithioalkyl) modified sugar moiety. For example, nucleotides positions 1 to 8 of repeat segment each comprises a 2′-O-methyldithiomethyl modified sugar moiety. In some embodiments, the repeat segment comprises, comprises about, comprises at least, comprises at least about, comprises at most, or comprises at most about, 0, 1, 2, 3, 4, 5, 6, 7, 8, 9, 10, 11, 12, 13, 14, 15, 16, 17, 18, 19, 20, 21, 22, 23, 24, 25, 26, 27, or a number or a range between any two of these values, nucleotides each comprising a 2′-O-methyldithiomethyl modified sugar moiety. For example, the repeat segment comprises 8 nucleotides each comprising a 2′-O-methyldithiomethyl modified sugar moiety.

In some embodiments, nucleotides at position 1, 2, 3, 4, 5, 6, 7, 8, 9, 10, 11, 12, 13, 14, 15, 16, 17, 18, 19, 20, 21, 22, 23, 24, 25, 26, and/or 27, of repeat segment each comprises a modified sugar moiety, such as a 2′-O-methyl or 2′-O-alkyl modified sugar moiety. For example, nucleotides at positions 9 to 12 of repeat segment each comprises a 2′-O-methyl modified sugar moiety. In some embodiments, repeat segment comprises, comprises about, comprises at least, comprises at least about, comprises at most, or comprises at most about, 0, 1, 2, 3, 4, 5, 6, 7, 8, 9, 10, 11, 12, 13, 14, 15, 16, 17, 18, 19, 20, 21, 22, 23, 24, 25, 26, 27, or a number or a range between any two of these values, nucleotides each comprising a modified sugar moiety, such as a 2′-O-methyl or 2′-O-alkyl modified sugar moiety. For example, repeat segment comprises nucleotides each comprises a 2′-O-methy modified sugar moiety.

### Tetra Loop

A tetraloop can be, can be about, can be at least, can be at least about, can be at most, or can be at most about, 3, 4, 5, 6, 7, 8, 9, or a number or a range between any two of these values, nucleotides in length. For example, tetraloop is 4 nucleotides or about 4 nucleotides in length. In some embodiments, nucleotides at position 1, 2, 3, 4, 5, 6, 7, 8, and/or 9, of tetraloop each comprises a modified sugar moiety, such as 2′-O-methyl or 2′-O-alkyl modified sugar moiety. For example, nucleotides at positions 1 to 4 of tetraloop each comprises a 2′-O-methyl modified sugar moiety. In some embodiments, tetra loop comprises, comprises about, comprises at least, comprises at least about, comprises at most, or comprises at most about, 0, 1, 2, 3, 4, 5, 6, 7, 8, 9, 10, 11, 12, 13, 14, 15, 16, 17, 18, 19, 20, 21, 22, 23, 24, 25, 26, 27, or a number or a range between any two of these values, nucleotides each comprising a modified sugar moiety, such as a 2′-O-methyl or 2′-O-alkyl modified sugar moiety. For example, tetraloop comprises 4 nucleotides each comprising a 2′-O-methy modified sugar moiety.

### Anti-Repeat

An anti-repeat segment can be, can be about, can be at least, can be at least about, can be at most, or can be at most about, 11, 12, 13, 14, 15, 16, 17, 18, 19, 20, 21, 22, 23, 24, 25, 26, 27, 28, 29, 30, 31, or a number or a range between any two of these values, in length. For example, anti-repeat segment is or is about 14 nucleotides in length.

In some embodiments, nucleotides at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and/or 31, of anti-repeat segment each comprises a 2′-O-methyldithiomethyl (or 2′-O-methyldithioalkyl) modified sugar moiety. For example, nucleotides at positions 5 to 14 of anti-repeat segment each comprises a 2′-O-methyldithiomethyl modified sugar moiety. For example, the last 10 nucleotides of anti-repeat segment each comprises a 2′-O-methyldithiomethyl modified sugar moiety. For example, the last, second last, third last, fourth last, fifth last, sixth last, seventh last, eighth last, ninth last, and/or tenth last nucleotides of anti-repeat segment each comprises a 2′-O-methyldithiomethyl (or 2′-O-methyldithioalkyl) modified sugar moiety. In some embodiments, anti-repeat segment comprises, comprises about, comprises at least, comprises at least about, comprises at most, or comprises at most about, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or a number or a range between any two of these values, nucleotides each comprising a 2′-O-methyldithiomethyl (or 2′-O-methyldithioalkyl). For example, anti-repeat segment comprises 10 nucleotides each comprising a 2′-O-methyldithiomethyl modified sugar moiety.

In some embodiments, nucleotides at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and/or 31, of anti-repeat segment each comprises a modified sugar moiety, such as a 2′-O-methyl or 2′-O-alkyl modified sugar moiety. For example, nucleotides at positions 1 to 4 of anti-repeat segment each comprises a 2′-O-methyl modified sugar moiety. For example, the first 4 nucleotides of anti-repeat segment each comprises a 2′-O-methyl modified sugar moiety. In some embodiments, anti-repeat segment comprises, comprises about, comprises at least, comprises at least about, comprises at most, or comprises at most about, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or a number or a range between any two of these values, nucleotides each comprising a modified sugar moiety, such as a 2′-O-methyl or 2′-O-alkyl modified sugar moiety. For example, anti-repeat segment comprises 4 nucleotides each comprising a 2′-O-methyl modified sugar moiety.

### Stem Loop 1

In some embodiments, stem loop 1 is, is about, is at least, is at least about, is at most, or is at most about, 7, 8, 9, 10, 11, 12, 13, 14, 15, 16, 17, 18, or a number or a range between any two of these values, nucleotides in length. For example, stem loop 1 is or is about 9 nucleotides in length. For example, stem loop 1 is or is about 11 nucleotides in length. For example, stem loop 1 is or is about 13 nucleotides in length.

In some embodiments, nucleotides at positions 1, 2, 3, 4, 5, 6, 7, 8, 9, 10, 11, 12, 13, 14, 15, 16, 17, and/or 18 of stem loop 1 each comprises a 2′-O-methyldithiomethyl (or 2′-O-methyldithioalky) modified sugar moiety. For example, nucleotides at positions 1 to 9 of stem loop 1 each comprises a 2′-O-methyldithiomethyl modified sugar moiety. For example, nucleotides at positions 1 to 11 of stem loop 1 each comprises a 2′-O-methyldithiomethyl modified sugar moiety. For example, nucleotides at positions 1 to 13 of stem loop 1 each comprises a 2′-O-methyldithiomethyl modified sugar moiety. In some embodiments, stem loop 1 comprises, comprises about, comprises at least, comprises at least about, comprises at most, or comprises at most about, 0, 1, 2, 3, 4, 5, 6, 7, 8, 9, 10, 11, 12, 13, 14, 15, 16, 17, 18, or a number or a range between any two of these values, nucleotides each comprising a 2′-O-methyldithiomethyl (or 2′-O-methyldithioalky) modified sugar moiety. For example, stem loop 1 comprises 9 nucleotides each comprises a 2′-O-methyldithiomethyl modified sugar moiety. For example, stem loop 1 comprises 11 nucleotides each comprises a 2′-O-methyldithiomethyl modified sugar moiety. For example, stem loop 1 comprises 13 nucleotides each comprises a 2′-O-methyldithiomethyl modified sugar moiety.

### Linker

A linker can be, can be about, can be at least, can be at least about, can be at most, or can be at most about, 1, 2, 3, 4, 5, 6, 7, 8, 9, 10, or a number or a range between any two of these values, nucleotides in length. For example, linker is 4 or 5 nucleotides or about 4 or about 5 nucleotides in length. In some embodiments, nucleotides at positions 1, 2, 3, 4, 5, 6, 7, 8, 9, and/or 10 of linker each comprises a 2′-O-methyldithiomethyl modified sugar moiety. For example, nucleotides at positions 1 to 4, or 1 to 5, of linker each comprises a 2′-O-methyldithiomethyl modified sugar moiety. In some embodiments, linker comprises, comprises about, comprises at least, comprises at least about, comprises at most, or comprises at most about, 0, 1, 2, 3, 4, 5, 6, 7, 8, 9, 10, or a number or a range between any two of these values, nucleotides each comprising a 2′-O-methyldithiomethyl modified sugar moiety. For example, linker can comprise 4 or 5 nucleotides each comprises a 2′-O-methyldithiomethyl modified sugar moiety.

### Stem Loop 2

In some embodiments, stem loop 2 is, is about, is at least, is at least about, is at most, or is at most about, 7, 8, 9, 10, 11, 12, 13, 14, 15, 16, 17, 18, 19, or a number or a range between any two of these values, nucleotides in length. For example, stem loop 2 is 12 nucleotides or about 12 nucleotides in length. For example, stem loop 2 is 14 nucleotides or about 14 nucleotides in length.

In some embodiments, nucleotides at positions 1, 2, 3, 4, 5, 6, 7, 8, 9, 10, 11, 12, 13, 14, 15, 16, 17, 18, and/or 19 of stem loop 2 each comprises a modified sugar moiety, such as a 2′-O-methyl or 2′-O-alky modified sugar moiety. For example, nucleotides at positions 1 to 12 of stem loop 2 each comprises a 2′-O-methyl modified sugar moiety. For example, nucleotides at positions 1 to 14 of stem loop 2 each comprises a 2′-O-methyl modified sugar moiety. In some embodiments, stem loop 2 comprises, comprises about, comprises at least, comprises at least about, comprises at most, or comprises at most about, 0, 1, 2, 3, 4, 5, 6, 7, 8, 9, 10, 11, 12, 13, 14, 15, 16, 17, 18, 19, or a number or a range between any two of these values, nucleotides each comprising a modified sugar moiety, such as a 2′-O-methylor 2′-O-alky modified sugar moiety. For example, stem loop 2 comprises 12 nucleotides each comprising a 2′-O-methyl modified sugar moiety. For example, stem loop 2 comprises 14 nucleotides each comprising a 2′-O-methyl modified sugar moiety.

### Stem Loop 3

In some embodiments, stem loop 3 is, is about, is at least, is at least about, is at most, or is at most about, 7, 8, 9, 10, 11, 12, 13, 14, 15, 16, 17, 18, 19, 20, or a number or a range between any two of these values, nucleotides in length. For example, stem loop 3 is 15 nucleotides or about 15 nucleotides in length.

In some embodiments, nucleotides at positions 1, 2, 3, 4, 5, 6, 7, 8, 9, 10, 11, 12, 13, 14, 15, 16, 17, 18, 19, and/or 20 of stem loop 3 each comprises a modified sugar moiety, such as a 2′-O-methyl or 2′-O-alky modified sugar moiety. For example, nucleotides at positions 1 to 15 of stem loop 3 each comprises a 2′-O-methyl modified sugar moiety. In some embodiments, stem loop 3 comprises, comprises about, comprises at least, comprises at least about, comprises at most, or comprises at most about, 0, 1, 2, 3, 4, 5, 6, 7, 8, 9, 10, 11, 12, 13, 14, 15, 16, 17, 18, 19, 20, or a number or a range between any two of these values, nucleotides each comprising a modified sugar moiety, such as a 2′-O-methylor 2′-O-alky modified sugar moiety. For example, stem loop 3 comprises 15 nucleotides each comprising a 2′-O-methyl modified sugar moiety.

### Terminator

A terminator segment can be, can be about, can be at least, can be at least about, can be at most, or can be at most about, 1, 2, 3, 4, 5, 6, 7, 8, 9, 10, or a number or a range between any two of these values, nucleotides in length. For example, terminator segment is 4 nucleotides or about 4 nucleotides in length.

In some embodiments, nucleotides at positions 1, 2, 3, 4, 5, 6, 7, 8, 9, and/or 10, terminator segment each comprises a modified sugar moiety, such as a 2′-O-methyl or 2′-O-alky modified sugar moiety. For example, nucleotides at positions 1 to 4 of terminator segment each comprises a 2′-O-methyl modified sugar moiety. In some embodiments, terminator segment comprises, comprises about, comprises at least, comprises at least about, comprises at most, or comprises at most about, 0, 1, 2, 3, 4, 5, 6, 7, 8, 9, 10, or a number or a range between any two of these values, nucleotides each comprising a modified sugar moiety, such as a 2′-O-methylor 2′-O-alky modified sugar moiety. For example, terminator segments comprises 4 nucleotides each comprising a 2′-O-methyl modified sugar moiety.

### Segments

In some embodiments, an RNA (e.g., a sgRNA) or a segment of the RNA (e.g., guide segment, repeat segment, tetraloop, anti-repeat segment, stem loop 1, linker, stem loop 2, stem loop 3, or terminator segment) is, is about, is at least, is at least about, is at most, or is at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, or a number or a range between any two of these values, nucleotides in length. For example, a sgRNA can be or be about 100 nucleotides, in length.

In some embodiments, any nucleotide of an RNA (e.g., sgRNA) or a segment of the RNA can comprise a 2′-O-methyldithiomethyl (or a 2′-O-methyldithioalkyl) modified sugar moiety. For example, nucleotides at positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, and/or 110 of an RNA (e.g., sgRNA) or a segment of the RNA can comprise a 2′-O-methyldithiomethyl (or a 2′-O-methyldithioalkyl) modified sugar moiety.

In some embodiments, any nucleotide of an RNA (e.g., sgRNA) or a segment of the RNA can comprise a modified sugar moiety that is not a 2′-O-methyldithiomethyl (or a 2′-O-methyldithioalkyl) modified sugar moiety. Such a modified sugar moiety can comprise a 2′-O-methyl (or a 2′-O-alkyl) modified sugar moiety. For example, nucleotides at positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, and/or 110 of an RNA (e.g., sgRNA) or a segment of the RNA can comprise a modified sugar moiety, such as a 2′-O-methyl (or a 2′-O-alkyl) modified sugar moiety.

In some embodiments, any nucleotide of an RNA (e.g., sgRNA) or a segment of the RNA can comprise a sugar moiety comprising 2′-OH. For example, nucleotides at positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, and/or 110 of an RNA (e.g., sgRNA) or a segment of the RNA can comprise a sugar moiety comprising 2′-OH.

In some embodiments, any nucleotide of an RNA (e.g., sgRNA) or a segment of the RNA can comprise a sugar moiety that is not modified. For example, nucleotides at positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, and/or 110 of an RNA (e.g., sgRNA) or a segment of the RNA can comprise a sugar moiety that is not modified.

In some embodiments, any nucleotide of an RNA (e.g., sgRNA) or a segment of the RNA can comprise a backbone modification. For example, nucleotides at positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, and/or 110 of an RNA (e.g., sgRNA) or a segment of the RNA can comprise a backbone modification.

In some embodiments, any nucleotide of an RNA (e.g., sgRNA) or a segment of the RNA can comprise no backbone modification. For example, nucleotides at positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, and/or 110 of an RNA (e.g., sgRNA) or a segment of the RNA can comprise no backbone modification.

In some embodiments, the sequence of the sgRNA comprises the sequence of

NNNNNNNNNNNNNNNNNNNN

GUUUUAGAGCUAGAAAUAGCAAGUUAAAAUAAGGCUAGUCCGUUAUCAACUU GAAAAAGUGGCACCGAGUCGGUGCUUUU (SEQ ID NO: 3). In some embodiments, the sgRNA comprises a sequence that is, is about, is at least, is at least about, is at most, or is at most, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values, identical to SEQ ID NO: 3. For example, the sgRNA comprises a sequence that is at least 90% identical to SEQ ID NO: 3. In some embodiments, the sgRNA comprises a sequence with, with about, with at least, with at least about, with at most, or with at most about, 0, 1, 2, 3, 4, 5, 6, 7, 8, 9, 10, 11, 12, 13, 14, 15, 16, 17, 18, 19, 20, or a number or a range between any two of these values, deletions relative to SEQ ID NO: 3. In some embodiments, the sgRNA comprises a sequence with, with about, with at least, with at least about, with at most, or with at most about, 0, 1, 2, 3, 4, 5, 6, 7, 8, 9, 10, 11, 12, 13, 14, 15, 16, 17, 18, 19, 20, or a number or a range between any two of these values, insertions relative to SEQ ID NO: 3. In some embodiments, the sgRNA comprises a sequence with, with about, with at least, with at least about, with at most, or with at most about, 0, 1, 2, 3, 4, 5, 6, 7, 8, 9, 10, 11, 12, 13, 14, 15, 16, 17, 18, 19, 20, or a number or a range between any two of these values, substitutions relative to SEQ ID NO: 3.

In some embodiments, an RNA or a segment of the RNA (e.g., guide segment, repeat segment, tetraloop, anti-repeat segment, stem loop 1, linker, stem loop 2, stem loop 3, or terminator segment) comprises a sequence that is, is about, is at least, is at least about, is at most, or is at most, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values, identical to SEQ ID NO: 3 or a subsequence thereof. For example, an RNA or a segment of the RNA comprises a sequence that is at least 90% identical to SEQ ID NO: 3 or a subsequence thereof. The subsequence can be, be about, be at least, be at least about, be at most, be at most about, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80, nucleotides in length. The subsequence can begin at nucleotide of position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, or 97, of SEQ ID NO: 3. The subsequence can end at nucleotide of position 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, of SEQ ID NO: 3.

In some embodiments, an RNA or a segment of the RNA comprises a sequence with, with about, with at least, with at least about, with at most, or with at most about, 0, 1, 2, 3, 4, 5, 6, 7, 8, 9, 10, 11, 12, 13, 14, 15, 16, 17, 18, 19, 20, or a number or a range between any two of these values, deletions relative to SEQ ID NO: 3 or a subsequence thereof. In some embodiments, a RNA or a segment thereof comprises a sequence with, with about, with at least, with at least about, with at most, or with at most about, 0, 1, 2, 3, 4, 5, 6, 7, 8, 9, 10, 11, 12, 13, 14, 15, 16, 17, 18, 19, 20, or a number or a range between any two of these values, insertions relative to SEQ ID NO: 3 or a subsequence thereof. In some embodiments, an RNA or a segment of a gRNA comprise a sequence with, with about, with at least, with at least about, with at most, or with at most about, 0, 1, 2, 3, 4, 5, 6, 7, 8, 9, 10, 11, 12, 13, 14, 15, 16, 17, 18, 19, 20, or a number or a range between any two of these values, substitutions relative to SEQ ID NO: 3 or a subsequence thereof.

In some embodiments, the variable region comprises the sequence of nucleotides at positions 1 to 20 of SEQ ID NO: 3. Guide segment can comprise the sequence of nucleotides at positions 1 to 20 of SEQ ID NO: 3. In some embodiments, the invariable region comprises the sequence of nucleotides at positions 21 to 100 of SEQ ID NO: 3. The invariable region can comprise a sequence that is at least 90% identical to positions 21 to 100 SEQ ID NO: 3. The invariable region can comprise one or more deletions, one or more insertions, and/or one or more substitutions relative to the sequence of positions 21 to 100 SEQ ID NO: 3.

Repeat segment can comprise the sequence of nucleotides at positions 21 to 32 of SEQ ID NO: 3. Tetraloop can comprise a sequence with at least 90% sequence identity to the sequence at positions 33 to 36 of SEQ ID NO: 3. Tetraloop can comprise one or more insertions, one or more deletions, and/or one or more substitutions relative to the sequence at positions 33 to 36 of SE ID NO: 3.

Anti-repeat segment can comprise the sequence of nucleotides at positions 37 to 50 of SEQ ID NO: 3. Anti-repeat can comprise a sequence with at least 90% sequence identity to the sequence at positions 37 to 50 of SEQ ID NO: 3. Anti-repeat can comprise one or more insertions, one or more deletions, and/or one or more substitutions relative to the sequence at positions 37 to 50 of SE ID NO: 3.

Stem loop 1 can comprise the sequence of nucleotides at positions 53 to 61, 52 to 62, or 51 to 63, of SEQ ID NO: 3. Stem loop 1 can comprise a sequence with at least 90% sequence identity to the sequence at positions 53 to 61, 52 to 62, or 51 to 63, of SEQ ID NO: 3. Stem loop 1 can comprise one or more insertions, one or more deletions, and/or one or more substitutions relative to the sequence at positions 53 to 61, 52 to 62, or 51 to 63, of SE ID NO: 3.

Linker can comprise the sequence of nucleotides at positions 63 to 67, or 64 to 67, of SEQ ID NO: 3. Linker can comprise a sequence with at least 90% sequence identity to the sequence at positions 63 to 67, or 64 to 67, of SEQ ID NO: 3. Linker can comprise one or more insertions, one or more deletions, and/or one or more substitutions relative to the sequence at positions 63 to 67, or 64 to 67, of SE ID NO: 3.

Stem loop 2 can comprise the sequence of nucleotides at positions 69 to 80, or 68 to 81, of SEQ ID NO: 3. Stem loop 2 can comprise a sequence with at least 90% sequence identity to the sequence at positions 69 to 80, or 68 to 81, of SEQ ID NO: 3. Stem loop 2 can comprise one or more insertions, one or more deletions, and/or one or more substitutions relative to the sequence at positions 69 to 80, or 68 to 81, of SE ID NO: 3.

Stem loop 3 can comprise the sequence of nucleotides at positions 82 to 96 of SEQ ID NO: 3. Stem loop 3 can comprise a sequence with at least 90% sequence identity to the sequence at positions 82 to 96 of SEQ ID NO: 3. Stem loop can comprise one or more insertions, one or more deletions, and/or one or more substitutions relative to the sequence at positions 82 to 96 of SE ID NO: 3.

Terminator segment can comprise the sequence of nucleotides at positions 97 to 100 of SEQ ID NO: 3. Terminator segment can comprise a sequence with at least 90% sequence identity to the sequence at positions 97 to 100 of SEQ ID NO: 3. Terminator segment can comprise one or more insertions, one or more deletions, and/or one or more substitutions relative to the sequence at positions 97 to 100 of SE ID NO: 3.

In some embodiments, guide segment is immediately 5′ to repeat segment. Repeat segment can be immediately 5′ to tetraloop. Tetraloop can be immediately 5′ to anti-repeat segment. Anti-repeat segment can be immediately 5′ to stem loop 1. Stem loop 1 can be immediately 5′ to linker. Linker can be immediately 5′ to stem loop 2. Stem loop 2 can be immediately 5′ to stem loop 3. Stem loop 3 can be immediately 5′ to terminator segment.

In some embodiments, guide segment and repeat segment are separated by at least one nucleotide, such as 2, 3, 4, 5, 6, 7, 8, 9, 10, or more or fewer nucleotides. Repeat segment and tetraloop can be separated by at least one nucleotide, such as 2, 3, 4, 5, 6, 7, 8, 9, 10, or more or fewer nucleotides. Tetraloop and anti-repeat segment can be separated by at least one nucleotide, such as 2, 3, 4, 5, 6, 7, 8, 9, 10, or more or fewer nucleotides. Anti-repeat segment and stem loop 1 can be separated by at least one nucleotide. Stem loop 1 and linker can be separated by at least one nucleotide, such as 2, 3, 4, 5, 6, 7, 8, 9, 10, or more or fewer nucleotides. Linker and stem loop 2 can be separated by at least one nucleotide, such as 2, 3, 4, 5, 6, 7, 8, 9, 10, or more or fewer nucleotides. Stem loop 2 and stem loop 3 can be separated by at least one nucleotide, such as 2, 3, 4, 5, 6, 7, 8, 9, 10, or more or fewer nucleotides. Stem loop 3 and terminator segment can be separated by at least one nucleotide, such as 2, 3, 4, 5, 6, 7, 8, 9, 10, or more or fewer nucleotides.

### Internucleotide Linkages

In some embodiments, the internucleotide linkages between two nucleosides of guide segment comprises a phosphorothioate internucleotide linkage. For example, the internucleotide linkage between the nucleotides at positions 1 and 2, 2 and 3, 3 and 4, 4 and 5, 5 and 6, 6 and 7, 7 and 8, 8 and 9, 9 and 10, 10 and 11, 11 and 12, 12 and 13, 13 and 14, 14 and 15, 15 and 16, 16 and 17, 17 and 18, 18 and 19, 19 and 20, 20 and 21, 21 and 22, 22 and 23, 23 and 24, 24 and 25, 25 and 26, 26 and 27, 27 and 28, 28 and 29, and/or 29 and 30 can comprise a phosphorothioate internucleotide linkage. For example, the internucleotide linkage between nucleosides at positions 1 to 2 of guide segment can comprise a phosphorothioate internucleotide linkage. For example, the internucleotide linkages between nucleosides at positions 1 and 2, 2 and 3, and 3 and 4 of guide segment can comprise phosphorothioate internucleotide linkages.

The internucleotide linkage between two nucleotides of terminator segment can comprise a phosphorothioate internucleotide linkage. For example, the internucleotide linkage between the nucleotides at positions 1 and 2, 2 and 3, 3 and 4, 4 and 5, 5 and 6, 6 and 7, 7 and 8, 8 and 9, and/or 9 and 10 can comprise a phosphorothioate internucleotide linkage. The internucleotide linkage between the nucleotides at positions 1 and 2, 2 and 3, 3 and 4, 4 and 5, 5 and 6, 6 and 7, 7 and 8, 8 and 9, and/or 9 and 10 can comprise a phosphorothioate internucleotide linkage. For example, the internucleotide linkage between last and second last, second last and third last, third last and fourth last, fourth last and fifth last, fifth last and sixth last, sixth last and seventh last, seventh last and eighth last, eighth last and ninth last, and/or ninth last and tenth last, nucleotides can comprise a phosphorothioate internucleotide linkage. For example, the internucleotide linkage between the last two nucleotides of terminator segment can comprise a phosphorothioate internucleotide linkage. For example, the internucleotide linkages between the last four nucleosides of terminator segment can comprise phosphorothioate internucleotide linkages. For example, the internucleotide linkages between the all nucleosides of terminator segment can comprise phosphorothioate internucleotide linkages.

### GalNAC

The RNA can comprise (e.g., conjugated with) a N-Acetylgalactosamine (GalNAc) or a triantennary GalNAc on the 3′-end. The triantennary GalNAc can comprise the structure

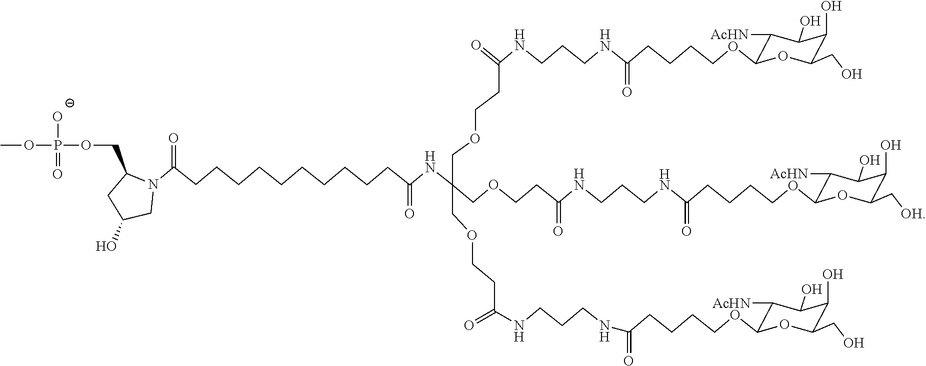 [SMILES: COP(=O)([O-])OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

Triantennary GalNAc can bind to the asioglycoprotein (ASGPR) receptor. Triantennary GalNAc can facilitate the targeted delivery of RNAs to hepatocytes. The RNA conjugated with a triantennary GalNAc can be administered subcutaneously without using a lipid nanoparticle (LNP).

### Base Modifications

In some embodiments, nucleobases at positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, and/or 110 of an RNA (e.g., sgRNA) or a segment of the RNA (e.g., guide segment, repeat segment, tetraloop, anti-repeat segment, stem loop 1, linker, stem loop 2, stem loop 3, or terminator segment) comprises a nucleobase analogue, such as an adenine nucleobase analogue, a cytosine nucleobase analogue, a guanine nucleobase analogue, or an uracil nucleobase analogue. In some embodiments, an RNA (e.g., sgRNA) or a segment of the RNA comprises, comprises about, comprises at least, comprises at least about, comprises at most, or comprises at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, or a number or a range between any two of these values, nucleobase analogues.

In some embodiments, the adenine nucleobase analogue comprises

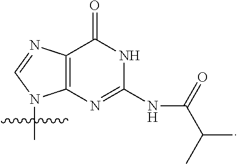

The cytosine nucleobase analogue can comprise

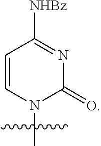

The guanine nucleobase analogue can comprise

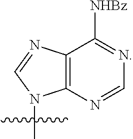

In some embodiments, a nucleobase analogue comprises 2-aminopurine, thymine, 2,6-diaminopurine, 2-pyrimidione, 5-methylcytosine, 5-hydroxymethylcytosine, hypoxanthine, xanthine, 7-methylguanine, or 5,6-dihydrouracil. In some embodiments, a nucleobase analog comprises 1-methyl-guanosine, 2,6-diaminopurine, 2-methyl-adenosine, 2-aminopurine, 4-thio-uridine, 5-bromo-uridine, 5-fluoro-cytidine, 5-fluoro-uridine, 5-iodo-uridine, 5-methyl-cytidine, 5-methyl-deoxycytidine, 5-methyl-uridine, inosine, n3-methyl-uridine, n6,n6-dimethyl-adenosine, n6-methyl-adenosine, pseudouridine, purine ribonucleoside, pyrrolo-cytidine, or ribavirin.

### Synthesis

Disclosed herein include methods of generating an RNA (e.g., gRNA or sgRNA) of the present disclosure. In some embodiments, a method of generating an RNA comprises chemically (e.g., using phosphoramidite chemistry) synthesizing a precursor nucleic acid comprising the sequence of the sgRNA using, for example, a synthesizer. A nucleotide with a 2′-O-methyldithiomethyl modified sugar moiety in the RNA can be synthesized using a nucleotide comprising a 2′-O-2,4,6-trimethoxybenzylthiomethyl (TMBTM) modified sugar moiety. A nucleotide with a 2′-O-methyldithiomethyl modified sugar moiety in the RNA can be synthesized using

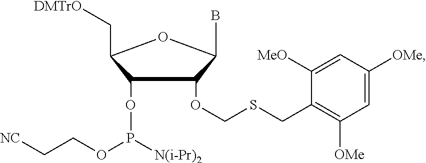

where B is the nucleobase of the nucleotide with the 2′-O-methyldithiomethyl modified sugar moiety (Scheme I). For example, a nucleotide with a 2′-O-methyldithiomethyl modified sugar moiety in the RNA can be synthesized using one of the following amidites.

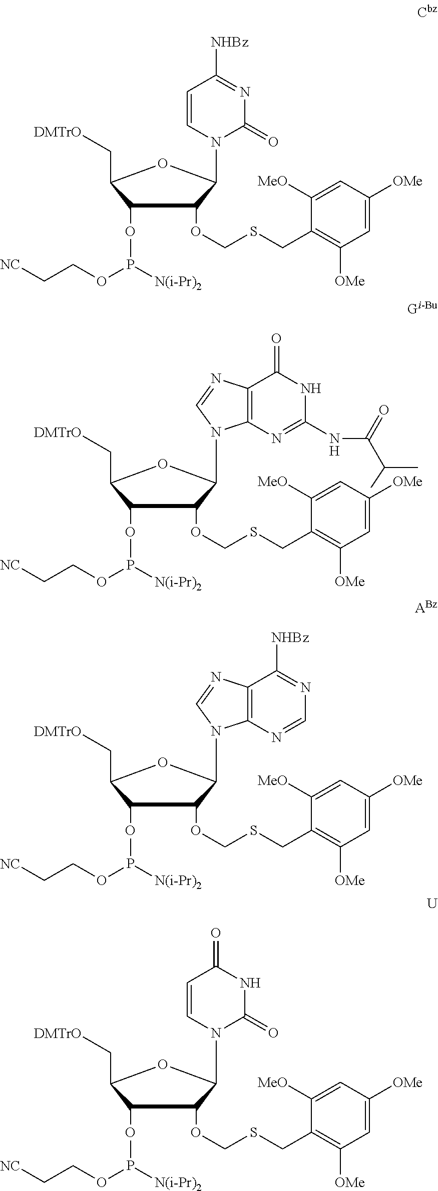

The amidine with the uracil nucleobase can be synthesized according to scheme II.

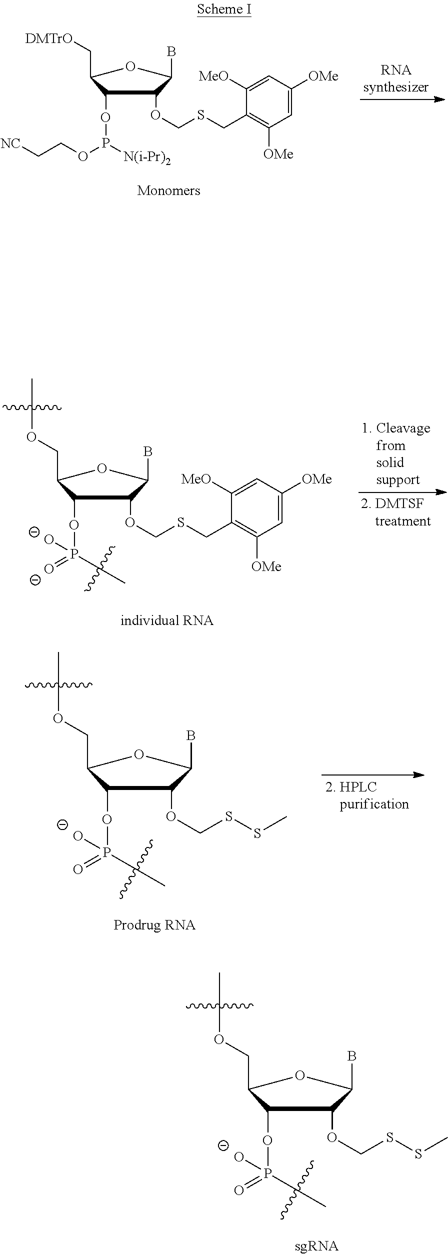

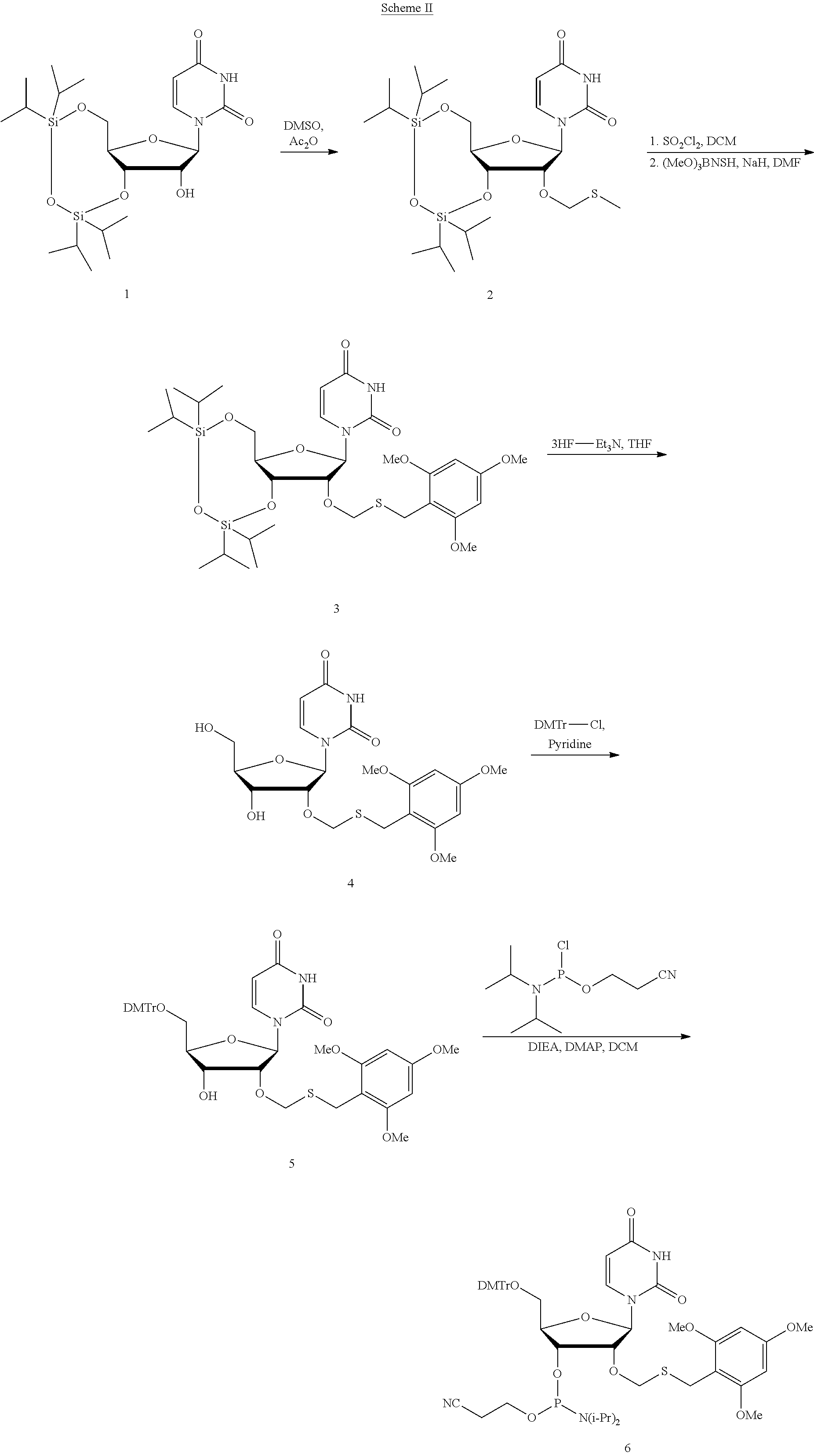

The method can comprise reacting a precursor nucleic acid with dimethyl(methylthio)-sulfonium tetrafluoroborate (DMTSF) (scheme I) to generate the RNA comprising the nucleotide with a 2′-O-methyldithiomethyl modified sugar moiety (referred to herein as prodrug RNA).

The reactions can be conducted under conditions sufficient to convert a precursor nucleic acid to the prodrug RNA. For example, in some embodiments, a precursor nucleic acid can be solubilized in an organic solvent comprising an alcohol (e.g., ethanol, isopropanol, or both) and dimethylsulfoxide (DMSO). In some embodiments, the organic solvent for solubilizing the precursor nucleic acid comprises ethanol and DMSO. The alcohol and DMSO can be combined at a molar ratio of about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, or a number or a range between any two of these values. In some embodiments, a high amount of ethanol and/or isopropanol in the precursor nucleic acid solution can reduce the formation of impurities such as adducts. Accordingly, the precursor nucleic acid can be solubilized in a solvent comprising an alcohol (e.g., ethanol, isopropanol, or both) and DMSO at a molar ratio greater than 1.

The DMTSF can also be solubilized in a solvent comprising DMSO, DMDS, ethanol, isopropanol, or a combination thereof. For example, in some embodiments, DMTSF can be solubilized in a solvent comprising DMSO and dimethyl disulfide (DMDS). The DMSO and DMDS can be combined in a molar ratio of about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10 or a number or a range between any two of these values. For example, the DMTSF can be solubilized in a solution comprising DMSO and DMDS in a molar ratio about 1:1. In some embodiments, an enhanced reaction rate can be achieved by minimizing or removing ethanol or isopropanol from the DMTSF solution.

In some embodiments, the precursor nucleic acid solubilized in a solution comprising DMSO and ethanol can be incubated with DMTSF in a solution comprising DMSO and DMDS for a time period (e.g., about 0.5-24 hours) suitable for generating the prodrug RNA.

The DMTSF treatment can be conducted at any suitable temperature. For example, the reactions are conducted at a temperature of, or a temperature about, 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C., or a number or a range between any two of these values. In some embodiments, the reactions is conducted at a temperature below 55° C. to reduce the degradation of the reaction product. The reaction can be conducted, for example, at or at about 25° C. In some embodiments, complete conversions is achieved by conducting the reaction at a temperature of 35° C. or less than 35° C.

The reactions can be conducted any suitable pH. For example, the reactions can be conducted at a pH of 4, 4.5, 5, 5.5, 6, or 6.5, or a number or a range between any two of these values. In some embodiments, the reaction is conducted at a pH of about 4 to about 6. The reactions can be conducted, for example, at a pH of from about 4 to about 4.5. In some embodiments, the reactions can be conducted at a pH of about 4.5 to avoid potential depurination.

The reactions can be conducted for any suitable length of time. In general, the reaction mixtures are incubated under suitable conditions for about 5 minutes to about several hours. The reactions can be conducted, for example, for about 5 minutes, 10 minutes, 20 minutes, 30 minutes, 1 hour, 1.5 hour, 2 hours, 3 hours, 4 hours, 6 hours, 8 hours, 10 hours, 12 hours, 14 hours, 16 hours, 18 hours, 20 hours, 22 hours, about 24 hours, or a number or a range between any two of these values. In some embodiments, the reactions are conducted for a period of time ranging from about 30 minutes to about 4 hours to reduce the amount of impurity product.

An RNA (e.g., a prodrug RNA) can be generated by ligating prodrug fragments (such as two or three fragments) using, for example, one or more splints (such as one or two splint). A splint can include sequences complementary to two prodrug fragments. A prodrug fragment can include a partial sequence of the RNA (e.g., gRNA or sgRNA). A prodrug fragment can include one or more 2′-O-methyldithiomethyl modified sugar moieties (scheme III). A precursor fragment comprising a nucleotide with 2′-O-2,4,6-trimethoxybenzylthiomethyl (TMBTM) modified sugar moiety can be synthesized (scheme III). With DMTSF treatment of the precursor fragment, the nucleotide with 2′-O-2,4,6-trimethoxybenzylthiomethyl modified sugar moiety is converted to a 2′-O-methyldithiomethyl modified sugar moiety in the resultant prodrug fragment (scheme III). The prodrug fragments can be ligated using splints to form the RNA (scheme III). A splint can include sequences complementary to two prodrug fragments. An RNA can be generated by ligating two fragments using a splint with sequences complementary to the two fragments. An RNA can be generated by ligating two fragments using two splints each with sequences complementary to two fragments. Generating an RNA (e.g., gRNA or sgRNA) by ligating RNA fragments using one or more splints has been described in U.S. Provisional Application No. 63/248,102, entitled “HIGH PURITY gRNA SYNTHESIS PROCESS,” filed Sep. 24, 2021; the content of which is incorporated herein in its entirety.

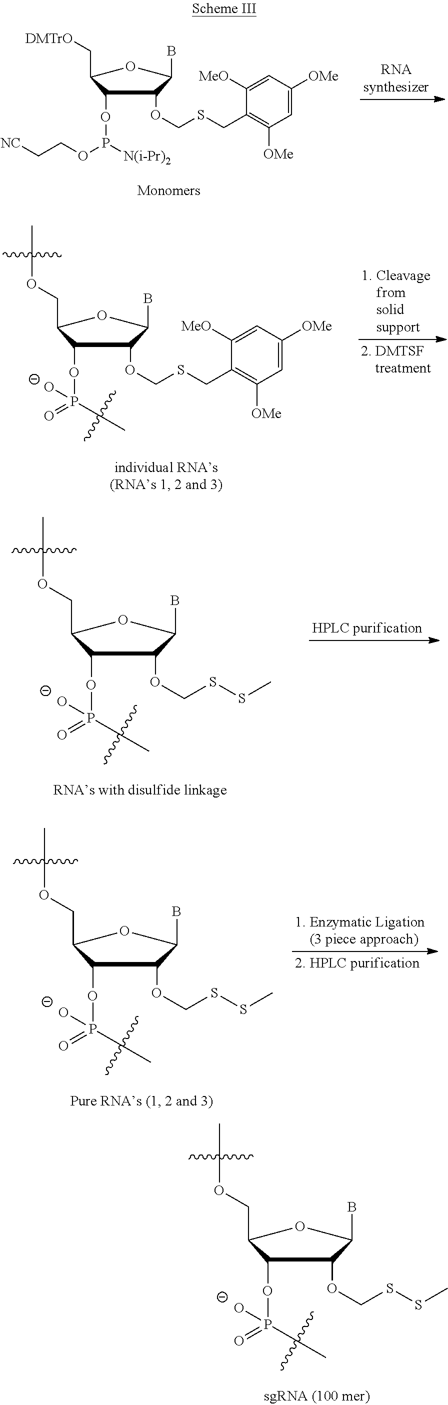

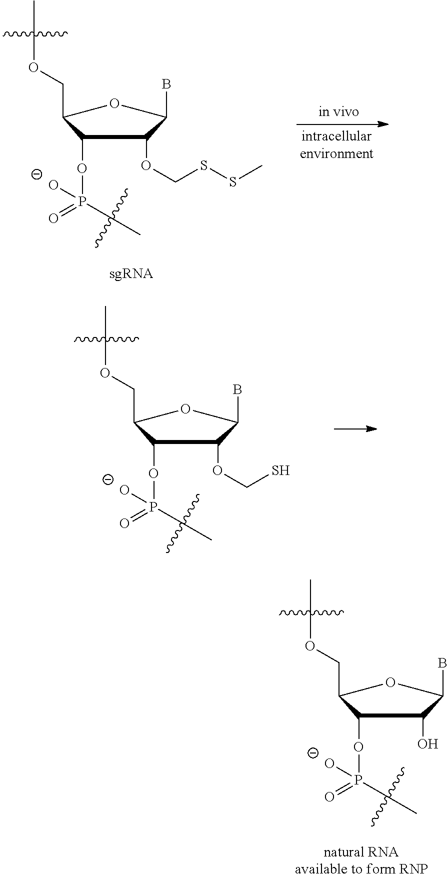

In some embodiments, a precursor nucleic acid is attached to a solid support. The method can comprise cleaving the precursor nucleic acid attached to the solid support to release the precursor nucleic acid from the solid support (scheme I, scheme III). In some embodiments, the method can comprise purifying the RNA generated. The RNA can be purified. The RNA can be purified high-performance liquid chromatography (HPLC) (scheme I). The RNA can be purified using ultra-filtration or with chromatographic methods. Non-limiting examples of chromatographic methods include: reversed-phase HPLC, ion-exchange chromatography (e.g., strong anion exchange HPLC or weak anion exchange HPLC), size exclusion chromatography, hydrophobic interaction chromatography, affinity chromatography, liquid chromatography-mass spectrometry (LCMS), capillary electrophoresis (CE), capillary gel electrophoresis (CGE), and polyacrylamide gel purification. In some embodiments, HPLC is used to purify the RNA. The HPLC columns can use a C18 substance as the stationary phase.

### Compositions

Disclosed herein include embodiments of a composition (e.g., a pharmaceutical composition). The composition can comprise an oligomeric compound, a gRNA, or a sgRNA of the present disclosure. In some embodiments, the composition comprises the oligomeric compound, gRNA, or sgRNA (e.g., with the reduction of the disulfide bonds of 2′-O-methyldithiomethyl groups of modified sugar moieties of the modified oligonucleotide of oligomeric compound, gRNA, or sgRNA, the 2′-O-methyldithiomethyl groups are converted 2′-O-methanethiol groups, which are then converted to 2′-OH) in complex with an RNA-guided endonuclease. The RNA-guided endonuclease can be a small Cas nuclease or a small RNA-guided endonuclease. The RNA-guided endonuclease can be a Cas9, a Cas12, aCas13, or a variant thereof. The RNA-guided endonuclease can be a Streptococcus pyogenes Cas9 (SpyCas9) or a Staphylococcus aureus Cas9 (SaCas9). The RNA-guided endonuclease can be a variant of Cas9. The variant of Cas9 can be a small Cas9, a dead Cas9 (dCas9), or a Cas9 nickase, The RNA-guided endonuclease can be a Campylobacter jejuni Cas9 (SpyCas9).

### Treatment

Disclosed herein include methods of treating a subject in need thereof. In some embodiments, a method of treating a subject in need thereof comprises administering an RNA (referred to herein as a prodrug RNA), such as a gRNA or sgRNA, or an oligomeric compound, or a composition thereof of the present disclosure to the subject. The method can comprise administering a lipid nanoparticle (LNP) comprising the RNA or the oligomeric compound. The RNA can be conjugated with a_triantennary GalNAc and be administered subcutaneously without using a LNP. The RNA can include one or more 2′-O-methyldithiomethyl modified sugar moieties. The RNA can be conditionally activatable. For example, in the reducing environment inside a cell of the subject, the disulfide bond of the 2′-O-methyldithiomethyl group of one (or one or more or each) modified sugar moiety of the administered RNA or oligomeric compound is reduced, forming a 2′-O-methanethiol group (scheme IV). The 2′-O-methanethiol group can in turn be converted (e.g., spontaneously converted) to 2′-OH (scheme IV). An RNA-guided endonuclease (e.g., Cas9) is able to bind the resultant RNA. The RNA can direct the activity of the RNA-guided endonuclease.

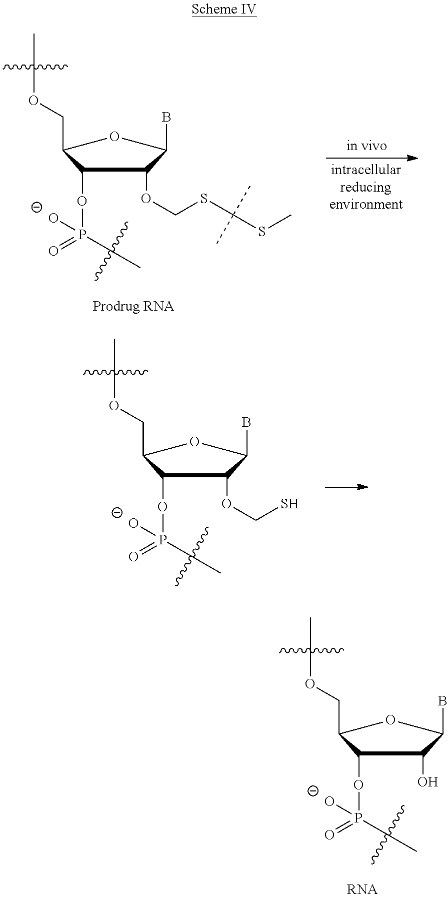

### RNA Modifications

An RNA fragment can include one or more modifications. Chemical modifications (include complete chemical modifications) may inhibit the activity of an RNA. Some positions need to be unprotected to form Cas9/sgRNA interaction effectively. The RNA fragment can include one or more modifications in the RNA backbone. Non-limiting examples of backbone modifications include: 2′ methoxy (2′OMe), 2′ fluorine (2′ fluoro), 2′-O-methoxy-ethyl (MOE), locked nucleic acids (LNA), unlocked nucleic acids (UNA), bridged nucleic acids, 2′ deoxynucleic acids (DNA), and peptide nucleic acids (PNA). Alternatively or additionally, the RNA fragment can include one or more base modifications. Non-limiting examples of base modifications include: 2-aminopurine, hypoxanthine, thymine, 2,6-diaminopurine, 2-pyrimidone, and 5-methyl cytosine. In some embodiments, a RNA fragment comprises at least one phosphorothioate linkage.

Modifications in the RNA fragment can be used to, for example, enhance stability, reduce the likelihood or degree of innate immune response, and/or enhance other attributes; and new types of modifications are regularly being developed. Non-limiting examples of modification include one or more nucleotides modified at the 2′ position of the sugar, such as but not limited to, a 2′-O-alkyl, 2′-O-alkyl-O-alkyl, or 2′-fluoro-modified nucleotide. DNA (2′ deoxy-) nucleotide substitutions are also contemplated. Non-limiting examples of RNA modifications also include 2′-fluoro, 2′-amino, 2′ O-methyl modifications on the ribose of pyrimidines, and basic residues or an inverted base at the 3′ end of the RNA. Such modifications can be incorporated into oligonucleotides, and these oligonucleotides have been shown to have a higher Tₘ (e.g., higher target binding affinity) than 2′-deoxy oligonucleotides against a given target. In some embodiments, the modification of an RNA fragment disclosed herein comprises a 2′-O-methyl modification of one or more nucleosides in the RNA fragment.

The RNA fragment can include one or more modifications that increase resistance to nuclease digestion as compared to the native nucleic acid. In some embodiments, the modified nucleic acid comprises a modified backbone selected from, for example, phosphorothioates, phosphotriesters, methyl phosphonates, short chain alkyl or cycloalkyl intersugar linkages, and short chain heteroatomic or heterocyclic intersugar linkages. The nucleic acid can have a phosphorothioate backbone or a heteroatom backbone, e.g., CH₂—NH—O—CH₂, CH, —N(CH₃)—O—CH₂ (known as a methylene(methylimino) or MMI backbone), CH₂—O—N(CH₃)—CH₂, CH₂—N(CH₃)—N(CH₃)—CH₂ and O—N(CH₃)—CH₂—CH₂ backbones; amide backbones; morpholino backbone structures; peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone. Phosphorus-containing linkages include, but are not limited to, phosphorothioates, chiral phosphorothioates, phosphorodithioates, phosphotriesters, aminoalkylphosphotriesters, methyl and other alkyl phosphonates including 3′ alkylene phosphonates and chiral phosphonates, phosphinates, phosphoramidates including 3′ amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. In some embodiments, the modification of the RNA fragment comprises one or more phosphorothioate linkages.

The RNA fragment can have a backbone that does not include a phosphorus atom, e.g., backbones that are formed by short chain alkyl or cycloalkyl internucleotide linkages, mixed heteroatom and alkyl or cycloalkyl internucleotide linkages, or one or more short chain heteroatomic or heterocyclic internucleotide linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

The RNA fragment can comprise one or more substituted sugar moieties including, one of the following at the 2′ position: OH, SH, SCH₃, F, OCN, OCH₃, OCH₃, O(CH₂)ₙ CH₃, O(CH₂)ₙ NH₂, or O(CH₂)ₙ CH₃, where n is from 1 to 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂, heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. For example, a modification can include 2′ methoxyethoxy (2′-O—CH₂CH₂OCH₃, also known as 2′-O-(2-methoxyethyl)). Other modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group. In some instances, both a sugar and an internucleotide linkage, e.g., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a PNA. In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. The RNA fragment can include 2′-O-thionocarbamates MP (2′-O-methyl-3′-phosphonoacetate) and/or MSP (O-methyl-3′-thiophosphonoacetate).

The RNA fragment can include one or more modifications selected from pseudouridine, N¹-methylpseudouridine, and 5-methoxyuridine. For example, one or more N¹-methylpseudouridine can be incorporated into the RNA fragment to provide enhanced RNA stability and reduced immunogenicity in animal cells, such as mammalian cells (e.g., cells of human and mice). N¹-methylpseudouridine modifications can also be incorporated in combination with one or more 5-methylcytidines.

The RNA fragment can include modifications designed to bypass innate antiviral responses and/or to reduce innate immune stimulation. For example, the RNA can be an enzymatically synthesized RNA incorporating 5′-Methylcytidine-5′-triphosphate (5-methyl-CTP), N6-methyl-ATP, pseudo-UTP, 2-thio-UTP, pseudoUTP, an Anti-Reverse Cap Analog (ARCA), or a combination thereof. The RNA fragment can include one or more modifications to enhance RNA stability, reduce innate immune responses, and/or achieve other benefits.

### Mimetics

The RNA fragment can be a nucleic acid mimetic. The term “mimetic” as it is applied to polynucleotides is intended to include polynucleotides wherein only the furanose ring or both the furanose ring and the internucleotide linkage are replaced with non-furanose groups. Replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone. In some embodiments, the RNA fragment is a PNA.

The RNA fragment can be a polynucleotide mimetic based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. Morpholino-based polynucleotides are nonionic mimics of oligonucleotides, which are less likely to form undesired interactions with cellular proteins. A variety of compounds within the morpholino class of polynucleotides have been prepared, having a variety of different linking groups joining the monomeric subunits.

The RNA fragment can be a polynucleotide mimetic referred to as cyclohexenyl nucleic acid (GeNA), where the furanose ring normally present in a DNA/RNA molecule is replaced with a cydohexenyl ring. GeNA DMT protected phosphoramidite monomers have been prepared and used for oligomeric compound synthesis following classical phosphoramidite chemistry.

The RNA fragment can be or comprise a locked nucleic acid (LNA), in which the 2′-hydroxyl group is linked to the 4′ carbon atom of the sugar ring, forming a 2′-C,4′-C-oxymethylene linkage, thereby forming a bicyclic sugar moiety. The linkage can be a methylene (—CH2-)ₙ group bridging the 2′ oxygen atom and the 4′ carbon atom wherein n is 1 or 2. LNA and LNA analogs display very high duplex thermal stabilities with complementary DNA and RNA (Tₘ=+3 to +10° C.), stability towards 3′-exonucleolytic degradation, and good solubility properties.

### Modified Sugar Moieties

An RNA fragment can include one or more substituted sugar moieties including, for example, a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly suitable are O((CH₂)ₙO)ₘCH₃, O(CH₂)ₙOCH₃, O(CH_{z})ₙNH₂, O(CH₂)CH₃, O(CH₂)ₙONH₂, and O(CH₂)ₙON((CH₂)ₙCH₃)₂, where n and m are from 1 to about 10. Other RNA fragments include a suitable sugar substituent group selected from: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, a group for improving the pharmacodynamic properties of an oligonucleotide, and other substituents having similar properties. A suitable modification includes 2′-methoxyethoxy 2′-O—CH₂—CH₂OCH₃, also known as −2′-O-(2-methoxyethyl) or 2′-MOE) (Martin et al. (1995) Helv. Chim. Acta, 78(2):486-504) e.g., an alkoxyalkoxy group. A further suitable modification includes 2′-dimethylaminooxyethoxy, e.g., a O(CH₂)₂ON(CH₃)₂ group, also known as 2′-DMAOE, as described in examples herein below, and 2′ dimethylaminoethoxyethoxy (also known in the art as 2′-O-dimethyl-amino-ethoxy-ethyl or 2′ DMAEOE), e.g., 2′-O—CH₂—O—CH₂—N(CH₃)₂.

Other suitable sugar substituent groups include methoxy (—O—CH₃), aminopropoxy (—O—CH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

The RNA fragment can include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentiobiosyl HMC, as well as synthetic nucleobases, e.g., 2-aminoadenine, 2-(methylamino)adenine, 2-(imidazolylalkyl)adenine, 2-(aminoalkylamino)adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl)adenine and 2,6-diaminopurine. A “universal” base known in the art, e.g. hypoxanthine, can also be included. 5-Me-C substitutions have been shown to increase nucleic acid duplex stability by 0.6-1.2° C. and are embodiments of base substitutions.

Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other a-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

The RNA fragment can include nucleobases for increasing the binding affinity. These include 5-substituted pyrimidines, 6-azapyrimidines and N-2, N-6 and —O-6 substituted purines, including 2-aminopropyladenine, 5-propynyluracil and 5-propynylcytosine. 5-methylcytosine substitutions have been shown to increase nucleic acid duplex stability by 0.6-1.2 oc.

The RNA fragment can comprise nucleobase modifications or substitutions may not have all positions uniformly modified. For example, an RNA fragment can have a modification incorporated in a single nucleoside.

### RNA Sequences and Properties

### Moderate Length RNAs

An exemplary RNA synthesized using any of the methods described herein can be a guide RNA (gRNA). A gRNA can be a single gRNA (sgRNA). A gRNA can comprise a CRISPR RNA (crRNA) or a trans-activating crRNA (tracrRNA). A gRNA provides target specificity by virtue of its association with the RNA-guided endonuclease, and thus directs the activity of the RNA-guided endonuclease. RNAs of the present disclosure can be synthesized from two or more RNA molecules (termed RNA fragments) using one or more splints. An exemplary double-molecule gRNA comprises a crRNA and a transactivating crRNA (tracrRNA), and the crRNA and tracrRNA hybridize to each other to form a duplex. A double-molecule gRNA can also be a duplex of two crRNAs. The gRNA duplex can bind a RNA-guided endonuclease such that the gRNA and the RNA-guided endonuclease form a complex. A crRNA comprises both a spacer sequence capable of hybridizing to a target nucleic acid sequence of interest and a crRNA repeat sequence. TracrRNAs can be in any form (e.g., full-length tracrRNAs or active partial tracrRNAs) and of varying lengths. For example, a tracrRNA may comprise or consist of all or a portion of a wild-type tracrRNA sequence (e.g., about or at least 20, 26, 32, 45, 48, 54, 63, 67, 85 or more nucleotides of a wild-type tracrRNA sequence). Examples of wild-type tracrRNA sequences from S. pyogenes include the 171-nucleotide, 89-nucleotide, 75-nucleotide, and 65-nucleotide versions. As an example, the crRNA can have, in the 5′ to 3′ direction, an optional spacer extension sequence, a spacer sequence, and a minimum CRISPR repeat sequence. The tracrRNA can have a minimum tracrRNA sequence (complementary to the minimum CRISPR repeat sequence), a 3′ tracrRNA sequence, and an optional tracrRNA extension sequence. The optional tracrRNA extension may have elements that contribute additional functionality (e.g., stability) to the gRNA, and can have one or more hairpin structures. The crRNA and the tracrRNA hybridize through the minimum CRISPR repeat sequence and the minimum tracrRNA sequence to form a gRNA.

An exemplary sgRNA comprises a nucleotide sequence that is complementary to a sequence in a target DNA, and a nucleotide sequence that can bind to an RNA-guided endonuclease. As an example, an sgRNA can have, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence, and an optional tracrRNA extension sequence. In some instances, an sgRNA can have, in the 5′ to 3′ direction, a minimum CRISPR repeat sequence and a spacer sequence. The single-molecule guide linker links the minimum CRISPR repeat and the minimum tracrRNA sequence to form a hairpin structure. In some embodiments, the single-molecule guide linker is a tetraloop.

A CRISPR repeat sequence can include any sequence that has sufficient complementarity with a tracr sequence to promote one or more of: (1) excision of a DNA targeting segment flanked by CRISPR repeat sequences in a cell containing the corresponding tracr sequence; and (2) formation of a CRISPR complex at a target sequence, wherein the CRISPR complex includes the CRISPR repeat sequence hybridized to the tracr sequence. In general, degree of complementarity is with reference to the optimal alignment of the CRISPR repeat sequence and tracr sequence, along the length of the shorter of the two sequences. Optimal alignment may be determined by any suitable alignment algorithm and may further account for secondary structures, such as self-complementarity within either the tracr sequence or CRISPR repeat sequence. In some instances, the degree of complementarity between the tracr sequence and CRISPR repeat sequence along the 30 nucleotides length of the shorter of the two when optimally aligned is about or more than 25%, 30%, 40%, 50%, 60%, 70%, 80%, 90%, 95%, 97.5%, 99%, or higher. The tracr sequence can be, or be about, 5, 6, 7, 8, 9, 10, 11, 12, 13, 14, 15, 16, 17, 18, 19, 20, 25, 30, 40, 50, or a number or a range between any two of these values, nucleotides in length.

The spacer of a gRNA includes a nucleotide sequence that is complementary to a sequence in a target DNA. In other words, the spacer of a gRNA interacts with a target DNA in a sequence-specific manner via hybridization (e.g., base pairing). As such, the nucleotide sequence of the spacer may vary and determines the location within the target DNA that the gRNA and the target DNA will interact. The spacer of a gRNA can be selected to hybridize to any desired sequence within a target DNA.

The spacer can have a length of 10, 11, 12, 13, 14, 15, 16, 17, 18, 19, 20, 21, 22, 23, 24, 25, 26, 27, 28, 29, 30, or a number or a range between any two of these values, nucleotides. For example, the spacer can have a length of from 13 to 25 nucleotides, from 15 to 23 nucleotides, from 18 to 22 nucleotides, or from 20 to 22 nucleotides.

The percent sequence complementarity between the spacer of a gRNA and a target sequence of a target DNA can be, e.g., at least about 60% (such as at least about any of 65%, 70%, 75%, 80%, 85%, 90%, 95%, 97%, 98%, 99%, or 100%).

The length of the gRNA synthesized by the methods described herein can be from 30 to 160 nucleotides, including 30, 35, 40, 45, 50, 55, 60, 65, 70, 75, 80, 85, 90, 95, 100, 105, 110, 115, 120, 125, 130, 135, 140, 145, 150, 155, 160, or a number or a range between any two of these values, nucleotides. The gRNA synthesized by the methods described herein can include a spacer. In some embodiments, the gRNA includes a sequence that is complementary to a sequence in a target DNA, including but not limited to, a target mammalian DNA. For example, the target DNA can be human DNA.

### Modifications in the RNAs

RNAs as described herein can include one or more modifications useful for e.g., tracking, increasing stability, targeting the RNA to a particular subcellular location, or reducing immunogenicity. Modifications of gRNAs can be used to enhance the formation or stability of a DNA-editing complex comprising a gRNA and an RNA-guided endonuclease (e.g., a Cas endonuclease, such as a Cas9 endonuclease). Modifications of gRNAs can also or alternatively be used to enhance the initiation, stability, or kinetics of interactions between a DNA-editing complex and a target sequence in a target DNA, which can be used, for example, to enhance on-target activity. Modifications of gRNAs can also or alternatively be used to enhance specificity, e.g., the relative rates of DNA editing at an on-target site as compared to effects at other (off-target) sites. Modifications can also or alternatively be used to increase the stability of an RNA, e.g., by increasing its resistance to degradation by ribonucleases (RNases) present in a cell, thereby causing its half-life in the cell to be increased.

The RNAs can include a segment at either the 5′ or 3′ end that provides for any of the features described above. For example, a suitable segment can include a riboswitch sequence (e.g., to allow for regulated stability and/or regulated accessibility by proteins and protein complexes); a stability control sequence; a sequence that forms a dsRNA duplex (e.g., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides for responses to light or radiation (e.g., UV, vis, IR optogenetic elements); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); a modification or sequence that provides for increased, decreased, and/or controllable stability; and combinations thereof.

The RNAs can include a modification that decreases the likelihood or degree to which the RNA, when introduced into a cell, elicits an innate immune response. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses. The RNAs can include one or more modifications selected from modifications that enhance the stability of the RNA (such as by decreasing its degradation by RNases, e.g., in the context of a cell) and modifications that decrease the likelihood or degree to which the RNA, when introduced into a cell, elicits an innate immune response. Combinations of modifications, such as the foregoing and others, can likewise be used.

### Stability Control Sequence

The RNAs can include a stability control sequence that influences the stability of the RNA. A non-limiting example of a suitable stability control sequence is a transcriptional terminator segment (e.g., a transcription termination sequence). A transcriptional terminator segment of an RNA can have a total length of from about 10 nucleotides to about 100 nucleotides, for example 10, 15, 20, 25, 30, 35, 40, 45, 50, 55, 60, 65, 70, 75, 80, 85, 90, 95, 100, or a number or a range between any two of these values, nucleotides in length.

The transcription termination sequence can be one that is functional in a eukaryotic cell, a prokaryotic cell, or both. Nucleotide sequences that can be included in a stability control sequence (e.g., transcriptional termination segment, or in any segment of the RNA to provide for increased stability) include, for example, a Rho-independent trp termination site.

### Conjugates

The RNAs can include a modification involving chemically linking to the gRNA one or more moieties or conjugates which enhance the activity, cellular distribution, or cellular uptake of the RNA. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a nucleic acid.

The RNAs can include a chemically linked conjugate moieties including, but not limited to, lipid moieties such as a cholesterol moiety; cholic acid; a thioether, e.g., hexyl-S-tritylthiol; a thiocholesterol; an aliphatic chain, e.g., dodecandiol or undecyl residues, a phospholipid, e.g. di-hexadecyl-rac-glycerol or triethylammonium 1,2-di-O-hexadecyl-rac-glycero-3-H phosphonate, a polyamine or a polyethylene glycol chain; adamantane acetic acid; a palmityl moiety; or an octadecylamine or hexylamino-carbonyl-t oxycholesterol moiety.

The RNAs can include a chemically linked conjugate including a “Protein Transduction Domain” or PTD (also known as a cell penetrating peptide, or CPP), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. A PTD can be covalently linked to a gRNA. Exemplary PTDs include, but are not limited to, a minimal undecapeptide protein transduction domain (corresponding to residues 47-57 of HIV-1 TAT); a polyarginine sequence comprising a number of arginines sufficient to direct entry into a cell (e.g., 3, 4, 5, 6, 7, 8, 9, 10, or 10-50 arginines); a VP22 domain; a Drosophila antennapedia protein transduction domain; a truncated human calcitonin peptide; and polylysine. The PTD can be an activatable CPP (ACPP) which includes a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane. The PTD can be chemically modified to increase the bioavailability of the PTD.

The RNAs can include an applied conjugate that can enhance its delivery and/or uptake by cells, including, for example, cholesterol, tocopherol and folic acid, lipids, peptides, polymers, linkers, and aptamers.

### RNA-Guided Endonucleases

As described herein, an RNA (e.g., a sgRNA) can comprise a sequence (or a portion of a sequence) that can bind to an RNA-guided endonuclease. The RNA-guided endonuclease can be naturally-occurring or non-naturally occurring. Non-limiting Examples of RNA-guided endonuclease include a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, and functional derivatives thereof. In some instances, the RNA-guided endonuclease is a Cas9 endonuclease. The Cas9 endonuclease can be from, e.g., Streptococcus pyogenes (SpyCas9), Staphylococcus lugdunensis (SluCas9), Staphylococcus aureus (SaCas9) and Campylobacter jejuni (CjCas9). In some embodiments, the RNA-guided endonuclease is a variant of Cas9, including but not limited to, a small Cas9, a dead Cas9 (dCas9), and a Cas9 nickase.

The RNA-guided endonuclease can be a small RNA-guided endonuclease. The small RNA-guided endonucleases can be engineered from portions of RNA-guided endonucleases derived from any of the RNA-guided endonucleases described herein and known in the art. The small RNA-guided endonucleases can be, e.g., small Cas endonucleases. In some cases, a small RNA-guided nuclease is shorter than about 1,100 amino acids in length.

The RNA-guided endonuclease can be a mutant RNA-guided endonuclease. For example, the RNA-guided endonuclease can be a mutant of a naturally occurring RNA-guided endonuclease. The mutant RNA-guided endonuclease can also be a mutant RNA-guided endonuclease with altered activity compared to a naturally occurring RNA-guided endonuclease, such as altered endonuclease activity (e.g., altered or abrogated DNA endonuclease activity without substantially diminished binding affinity to DNA). Such modification can allow for the sequence-specific DNA targeting of the mutant RNA-guided endonuclease for the purpose of transcriptional modulation (e.g., activation or repression); epigenetic modification or chromatin modification by methylation, demethylation, acetylation or deacetylation, or any other modifications of DNA binding and/or DNA-modifying proteins known in the art. In some embodiments, the mutant RNA-guided endonuclease has no DNA endonuclease activity.

The RNA-guided endonuclease can be a nickase that cleaves the complementary strand of the target DNA but has reduced ability to cleave the non-complementary strand of the target DNA, or that cleaves the non-complementary strand of the target DNA but has reduced ability to cleave the complementary strand of the target DNA. In some embodiments, the RNA-guided endonuclease has a reduced ability to cleave both the complementary and the non-complementary strands of the target DNA.

### EXAMPLES

Some aspects of the embodiments discussed above are disclosed in further detail in the following examples, which are not in any way intended to limit the scope of the present disclosure.

### Example 1

### Synthesis of Prodrug gRNA Segments

The example describes chemical procedures for synthesizing prodrug gRNA segments from precursor nucleic acids.

A mT2-RNA1 having a sequence of g*a*g*AACGCACCACUUUACGAGUUUUAGAgcuag (SEQ ID NO: 5; bold nucleotides are 2′-OMe nucleotides; * denotes a phosphorothioate internucleotide linkage) was synthesized at a scale of 15 μmol as DMT (dimethoxytrityl)-off oligos or DMT-on oligos. DMT-on oligos can be deprotected using any applicable strategy such as an ammonia treatment as will be understood by a skilled person (e.g., at 55° C. for about 9 or more hours)

TMB-oligos were synthesized from monomers comprising a 2′-O-2,4,6-trimethoxybenzylthiomethyl modified sugar moiety using a RNA synthesizer (see Scheme I).

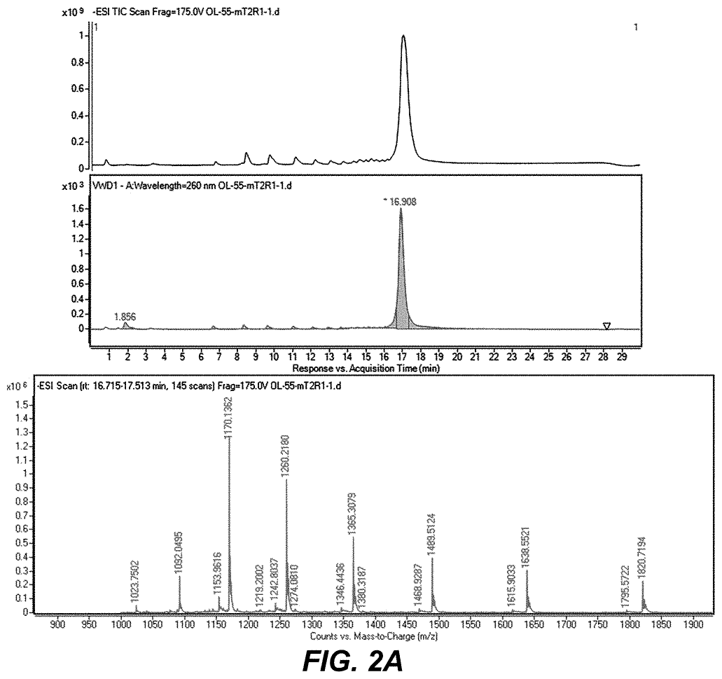

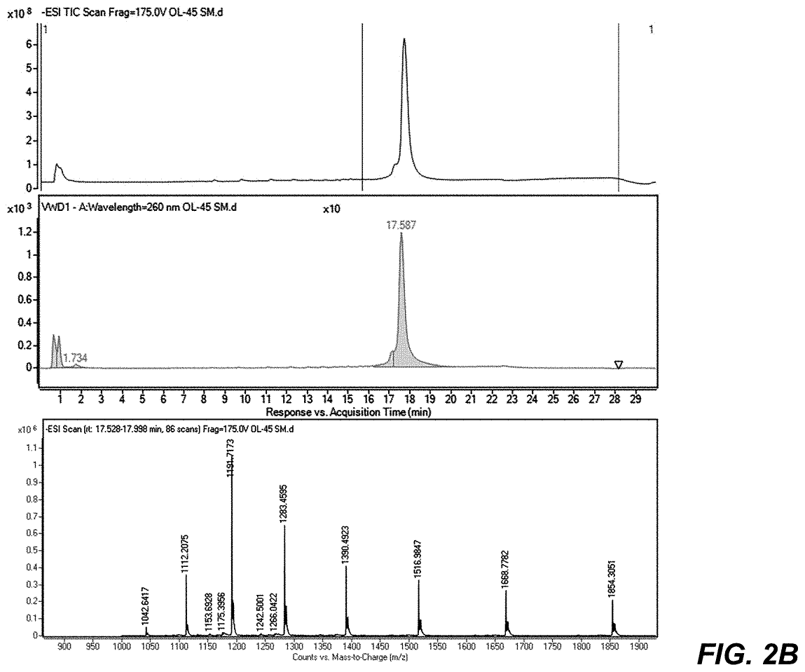

FIG. 2A-B are the HPLC/MS profiles of the TMB-oligos (i.e. oligos comprising a 2′-O-2,4,6-trimethoxybenzylthiomethyl modified sugar moiety) before DMTSF treatment with the DMT protecting group off (FIG. 2A) or on (FIG. 2B). In some embodiments, the DMT protecting group does not demonstrate apparent advantage in RNA purification for oligos containing multiple prodrug units due to the hydrophobic nature of the prodrug.

A number of trials were carried out for the DMTSF treatment of mT2-RNA1 solution. Table 1 provides the reaction conditions for each of the trials.

TABLE 1
DMTSF treatment trials

| Trial | 100 OD Oligo | 0.1M DMTSF | | |
|---|---|---|---|---|
| No. | solution | solution | Condition | LCMS result |
| 1.1 | 100 uL, in pH 4.0 | 100 uL, in pH 4.0 | 25° C., 4 h | Very broad and |
| | NaOAc buffer | NaOAc buffer | | heterogeneous peak, |
| 1.2 | 100 uL, in pH 4.0 | 100 uL, in pH 4.0 | 40° C., 2 h | with very minimal |
| | NaOAc buffer | NaOAc buffer | | degree of conversion |
| 1.3 | 100 uL, in pH 4.5 | 100 uL, in pH 4.5 | 25° C., 4 h | |
| | NaOAc buffer | NaOAc buffer | | |
| 1.4 | 100 uL, in pH 4.5 | 100 uL, in pH 4.5 | 40° C., 2 h | |
| | NaOAc buffer | NaOAc buffer | | |
| 2.1 | 40 uL, in | 80 uL, in 1:1 | 25° C., 18 h | Product, Product −93: |
| | DMSO | DMSO-DMDS | | intramolecular SS |
| 2.2 | 40 uL, in | 80 uL, in 1:1 | 55° C., 2 h | Product +180, |
| | DMSO | DMSO-DMDS | | (TMB adduct) |
| 2.3 | 40 uL, in | 80 uL, in 1:1 | 55° C., 16 h | Lot of desulfurization |
| | DMSO | DMSO-DMDS | | |
| 2.1 | 40 uL, in | 80 uL, in 1:1 | 25° C., 18 h | |
| | DMSO | DMSO-DMDS | | |
| 2.2 | 40 uL, in | 80 uL, in 1:1 | 25° C., 18 h | Water inhibits |
| | DMSO + 2 uL | DMSO-DMDS | | conversion |
| | water | | | |
| 2.3 | 40 uL, in | 80 uL, in 1:1 | 25° C., 18 h | EtOH reduces TMB |
| | DMSO + 2 uL | DMSO-DMDS | | adduct |
| | EtOH | | | |
| 3.1 | 50 uL, in | 70 uL, in 1:1 | 25° C., 18 h | |
| | DMSO + 20 uL | DMSO-DMDS | | |
| | DMSO | | | |
| 3.2 | 50 uL, in | 70 uL, in 1:1 | 25° C., 18 h | Higher amount of |
| | DMSO + 20 uL | DMSO-DMDS | | EtOH reduces TMB |
| | EtOH | | | adduct |
| 3.3 | 50 uL, in | 70 uL, in 1:1 | 25° C., 18 h | Pyrrolidone inhibits |
| | DMSO + 20 uL | DMSO-DMDS | | conversion |
| | pyrrolidone | | | |
| 4.1 | 50 uL, in | 100 uL, in 1:1 | 25° C., 18 h | Higher amount of |
| | DMSO + 50 uL | DMSO-DMDS | | EtOH reduces TMB |
| | EtOH | | | adduct |
| 4.2 | 50 uL, in | 100 uL, in 1:1 | 25° C., 18 h | Higher amount of |
| | DMSO + 100 uL | DMSO-DMDS | | EtOH reduces TMB |
| | EtOH | | | adduct |
| 5.1 | 50 uL, in | 100 uL, in 1:1:2 | 4 h, 24 h | EtOH in DMTSF |
| | DMSO + 50 uL | DMSO-DMDS-EtOH | | slows down reaction |
| | EtOH | | | |
| 5.2 | 50 uL, in | 100 uL, in 1:1 | 4 h, 24 h | Optimal conditions |
| | DMSO + 150 uL | DMSO-DMDS | | |
| | EtOH | | | |

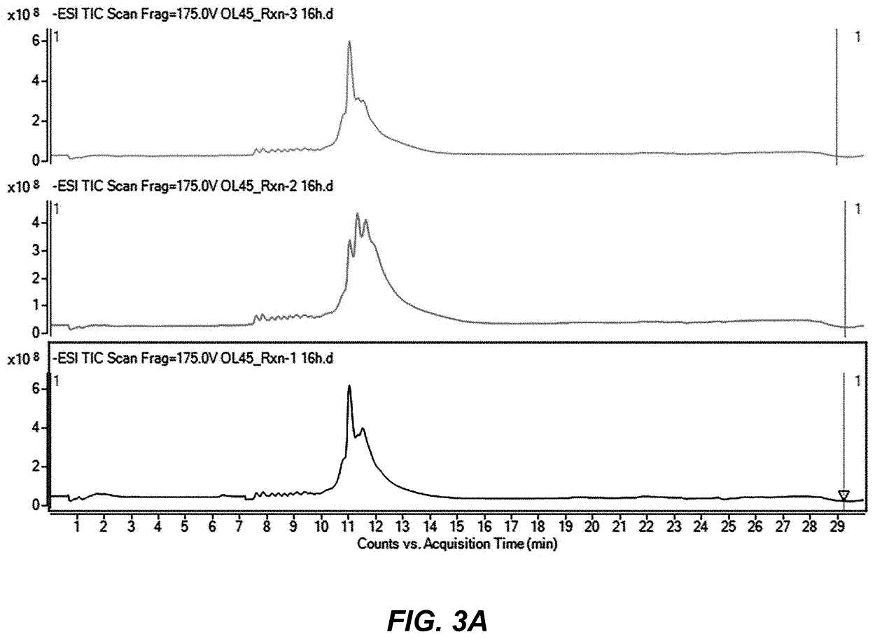

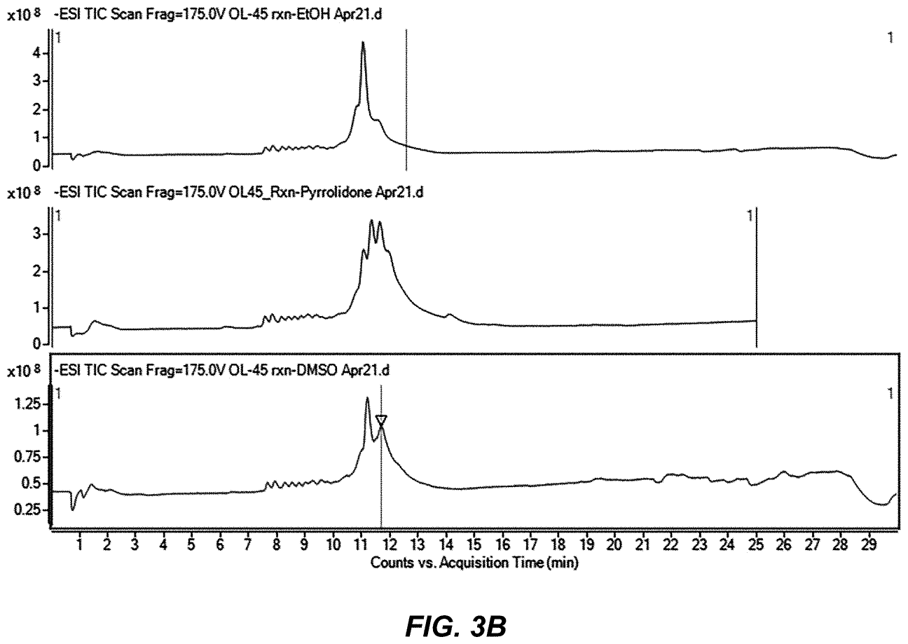

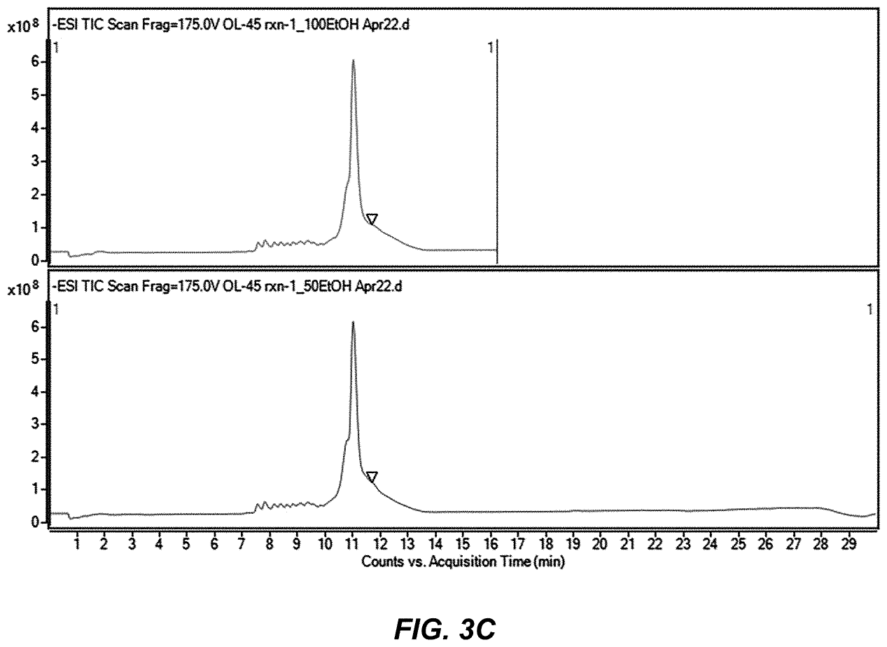

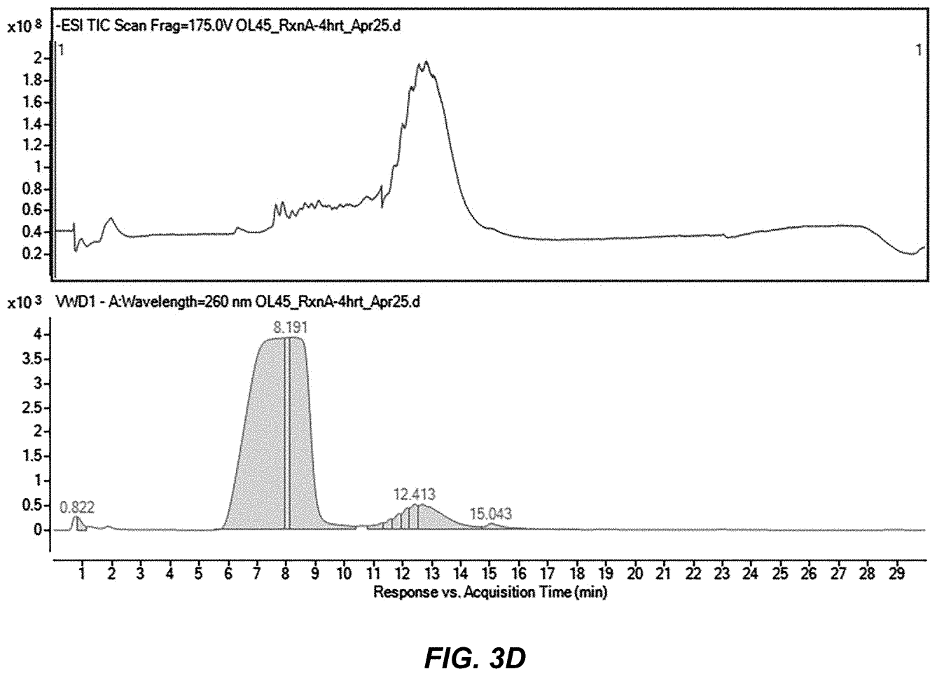

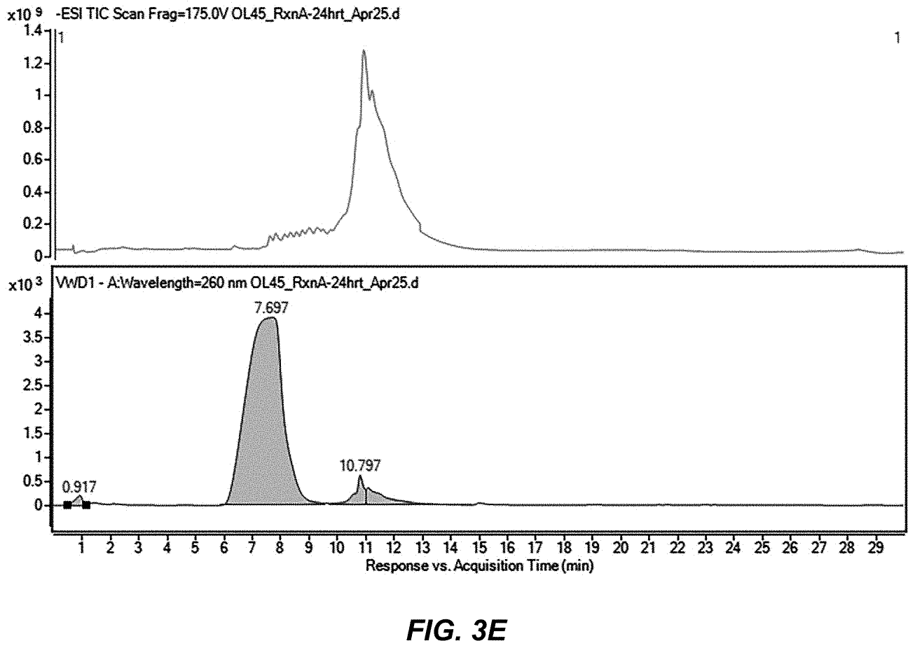

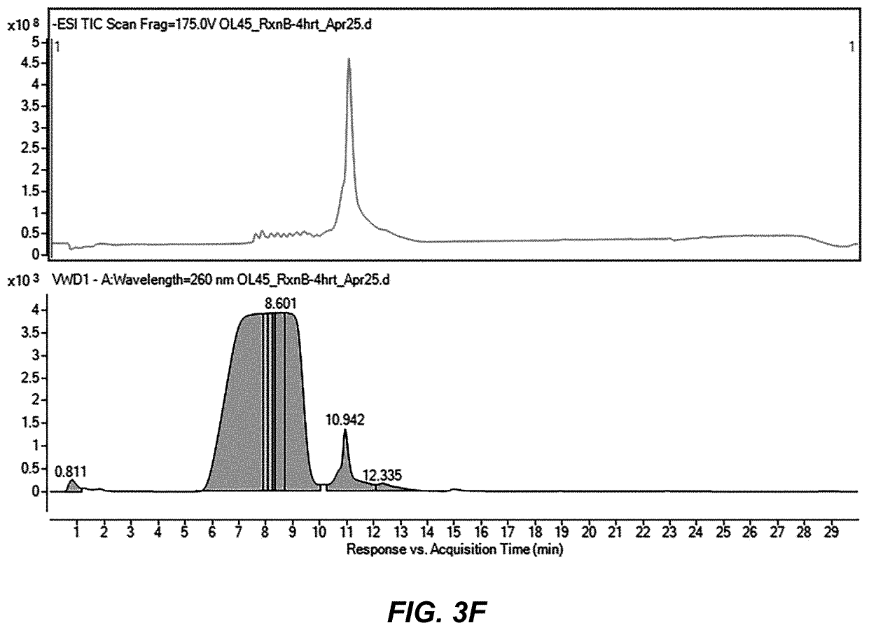

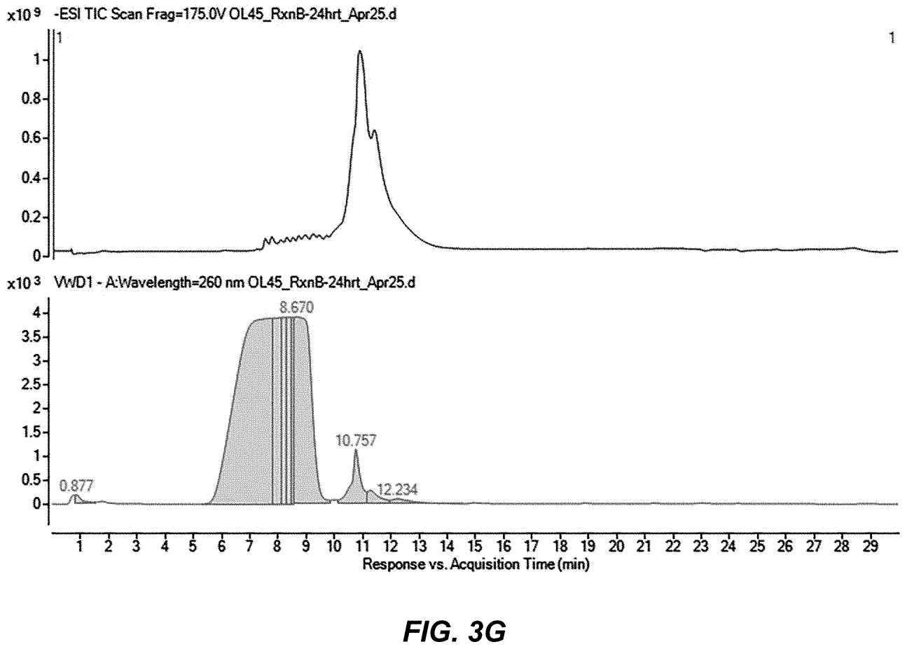

FIG. 3A-3G are the exemplary LCMS HPCL profiles of the synthesized oligonucleotides after the DMTSF treatment (FIG. 3A: trial 2.1, 2.2 with water and 2.3 with EtOH; FIG. 3B: trial 3.1, 3.2 and 3.3; FIG. 3C: trial 4.1 and 4.2; FIG. 3D: trial 5.1 with 4 h reaction time; FIG. 3E: trial 5.1 with 24 h reaction time; FIG. 3F: trial 5.2 with 4 h reaction time; and FIG. 3G: trial 5.2 with 24 h reaction time).

In some embodiments, water (e.g., 2% or less) or pyrrolidone in the oligo solution can reduce the conversion rate while ethanol can reduce the formation of TMB adduct. In some embodiments, ethanol in DMTSF solution can slow down the reaction. In some embodiments, longer reaction time leads to an increase of TMB adduct. In some embodiments, a reaction under pH about 4.5 can avoid potential depurination.

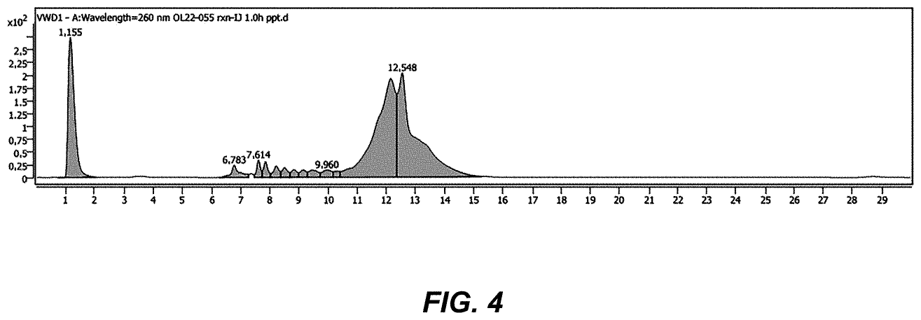

In some embodiments, a solution of TMB-oligos in 50% EtOH-DMSO (20 mg in 1.9 mL) was then treated with 0.3 mL of DMTSF solution in 50% DMSO-DMDS (0.5 M, 100 mg/mL) for 0.5-1.0 h. The reaction mixture was then diluted with 1.5 mL of water and precipitated with 10 mL of 3% LiClO₄ in acetone. The product was then purified using C18-HPLC. FIG. 4 is the LCMS HPCL profile after DMTSF treatment in a scale up version of trial 5.2 of Table 1.

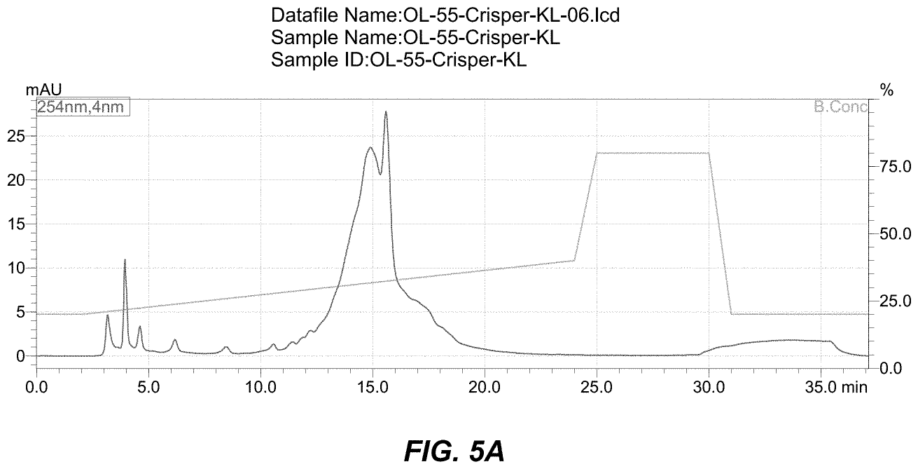

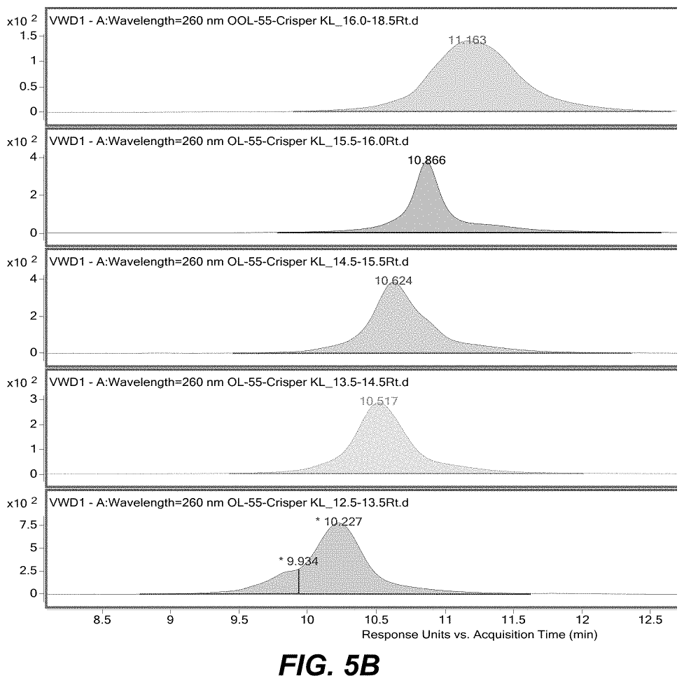

FIG. 5A is a HPLC purification profile of synthesized oligonucleotides. FIG. 5B shows the fraction analyses (RP-LCMS) of synthesized oligonucleotides: Fraction_16.0-18.5Rt: 1311.7 and 1321.0 ((M−Z)/Z, 10^{th} charge state); Fraction_15.5-16.0Rt: 1293.6 and 1303 ((M−Z)/Z, 10^{th} charge state); Fraction_14.5-15.5Rt: 1284.1, 1293.6 and 1303 ((M−Z)/Z, 10^{th} charge state); Fraction_13.5-14.5Rt: 1274.9, 1284.1 and 1293.6 ((M−Z)/Z, 10^{th} charge state); Fraction_12.5-13.5Rt: 1274.9 and 1284.1 ((M−Z)/Z, 10^{th} charge state). Impurities: <9.93 min. The synthesized mT2-RNA1 was measured at >500 ODU at 260 nm wavelength, corresponding to the fraction of 14.0-16.2 min.

Similar procedure was also used to synthesize mT2-RNA-2 having a sequence of PaaauagcAAGUUAAAAUAAGGCUAGUCCGUUAUCaacuu-3′ (SEQ ID NO: 6; bold and underlined nucleotides are 2′-O-Me nucleotides; and P at the 5′ indicates a phosphate).

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A single guide ribonucleic acid (sgRNA) according to the following formula:
5′—guide segment—repeat segment—tetraloop—anti-repeat segment—stem loop 1—linker—stem loop 2—stem loop 3—terminator segment −3′,
wherein:
guide segment, repeat segment, anti-repeat segment, stem loop 1, and/or linker comprises one or more nucleotides each comprising a 2′-O-methyldithiomethyl modified sugar moiety.

2. The sgRNA of claim 1, wherein:
guide segment, repeat segment, tetraloop, anti-repeat segment, stem loop 2, stem loop 3, and/or terminator segment comprises one or more nucleotides each comprising a modified sugar moiety and/or a modification in the backbone of the sgRNA.

3. The sgRNA of claim 2, wherein the modified sugar moiety comprises 2′-O-methyl (2′-Me), 2′ fluorine (2′-F), 2′-O-methoxy-ethyl (2′-MOE), a (S)-constrained ethyl (cEt), a locked nucleic acid (LNA), an unlocked nucleic acid (UNA), a bridged nucleic acid, an ethylene-bridged nucleic acid (ENA), or a deoxynucleic acid (DNA).

4. The sgRNA of claim 2, wherein the modification in the backbone of the sgRNA comprises a peptide nucleic acid (PNA), a phosphorothioate (PS) a phosphorodiamidate morpholino (PMO), a phosphoramidate, or a thiophosphoramidate.

5. The sgRNA of claim 1,
wherein:
guide segment, repeat segment, anti-repeat segment, stem loop 1, and/or linker comprises one or more nucleotides each comprising a 2′-O-methyldithiomethyl modified sugar moiety, and
guide segment, repeat segment, tetraloop, anti-repeat segment, stem loop 2, stem loop 3, and/or terminator segment comprises one or more nucleotides each comprising a 2′-O-methyl modified sugar moiety.

6. The sgRNA of claim 1, wherein:
the internucleotide linkages between two nucleosides of the guide segment comprises a phosphorothioate internucleotide linkage, optionally the internucleotide linkage between nucleosides at positions 1-2 comprise a phosphorothioate internucleotide linkage, optionally the internucleotide linkages between nucleosides at positions 1-4 comprise phosphorothioate internucleotide linkages, and
the internucleotide linkage between two nucleotides of terminator segment comprises a phosphorothioate internucleotide linkage, the internucleotide linkage between the last two nucleotides of terminator segment comprises a phosphorothioate internucleotide linkage, optionally the internucleotide linkages between the last four nucleosides of terminator segment comprise phosphorothioate internucleotide linkages.

7. The sgRNA of claim 1,
wherein:
nucleotides at positions 1-3 of guide segment comprise 2′-O-methyl modified sugar moieties,
nucleosides of tetraloop and the four 3′ most nucleotides of the repeat segment, and the four 5′ most nucleotides of the anti-repeat segment comprise 2′-O-methyl modified sugar moieties,
nucleotides of stem loop 2, stem loop 3, and terminator segment comprise 2′-O-methyl modified sugar moieties, and
the reminder nucleotides of the sgRNA comprise 2′-O-methyldithiomethyl modified sugar moieties, and
wherein:
the internucleotide linkages between nucleosides at positions 1-4 of guide segment comprise phosphorothioate internucleotide linkages, and
the internucleotide linkages between the last two to four nucleosides of terminator segment comprise phosphorothioate internucleotide linkages.

8. The sgRNA of claim 1, wherein guide segment is complementary to a sequence of a target, optionally the target is a mammalian target or a human target.

9. The sgRNA of claim 1, wherein guide segment comprises 20 nucleotides or about 20 nucleotides.

10. (canceled)

11. (canceled)

12. The sgRNA of claim 1, wherein repeat segment is 12 nucleotides or about 12 nucleotides in length, tetraloop is 4 nucleotides or about 4 nucleotides in length, anti-repeat segment is 14 nucleotides or about 14 nucleotides in length, stem loop 1 is 9 to 13 nucleotides or about 9 to 13 nucleotides in length, linker is 4 or 5 nucleotides or about 4 or about 5 nucleotides in length, stem loop 2 is 12 or 14 nucleotides or about 12 or 14 nucleotides in length, stem loop 3 is 15 nucleotides or about 15 nucleotides in length, and/or terminator segment is 4 nucleotides or about 4 nucleotides in length.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. The sgRNA of claim 1,
wherein guide segment comprises the sequence of nucleotides at positions 1 to 20 of the sequence of
(SEQ ID NO: 3)
nnnNNNNNNNNNNNNNNNNNGUUUUAGAgcuagaaauagcAAG
UUAAAAUAAGGCUAGUCCGUUAUCaacuugaaaaaguggca
ccgagucggugcuuuu,
wherein repeat segment comprises the sequence of nucleotides at positions 21 to 32 of SEQ ID NO: 3, and optionally with insertion(s), deletion(s), and/or substitution(s),
wherein tetraloop comprises the sequence of nucleotides at positions 33 to 36 of SEQ ID NO: 3, and optionally with insertion(s), deletion(s), and/or substitution(s),
wherein anti-repeat segment comprises the sequence of nucleotides at positions 37 to 50 of SEQ ID NO: 3, and optionally with insertion(s), deletion(s), and/or substitution(s),
wherein stem loop 1 comprises the sequence of nucleotides at positions 53 to 61, 52 to 62, or 51 to 63, of SEQ ID NO: 3, and optionally with insertion(s), deletion(s), and/or substitution(s),
wherein linker comprises the sequence of nucleotides at positions 63 to 67, or 64 to 67, of SEQ ID NO: 3, and optionally with insertion(s), deletion(s), and/or substitution(s),
wherein stem loop 2 comprises the sequence of nucleotides at positions 69 to 80, or 68 to 81, of SEQ ID NO: 3, and optionally with insertion(s), deletion(s), and/or substitution(s),
wherein stem loop 3 comprises the sequence of nucleotides at positions 82 to 96 of SEQ ID NO: 3, and optionally with insertion(s), deletion(s), and/or substitution(s), and/or
wherein terminator segment comprises the sequence of nucleotides at positions 97 to 100 of SEQ ID NO: 3, and optionally with insertion(s), deletion(s), and/or substitution(s),

31. The sgRNA of claim 30, wherein the sequence of the sgRNA comprises the sequence of SEQ ID NO: 3.

32. The sgRNA of claim 30, wherein the sgRNA comprises a nucleic acid sequence that is at least 90% identical to SEQ ID NO: 3, and/or wherein the gRNA comprises a nucleic acid sequence with one or more deletion(s), insertion(s), or substitution(s), relative to SEQ ID NO: 3.

33. The sgRNA of claim 1,
wherein guide segment and repeat segment are separated by at least one nucleotide,
repeat segment and tetraloop are separated by at least one nucleotide,
tetraloop and anti-repeat segment are separated by at least one nucleotide,
anti-repeat segment and stem loop 1 are separated by at least one nucleotide,
stem loop 1 and linker are separated by at least one nucleotide,
linker and stem loop 2 are separated by at least one nucleotide,
stem loop 2 and stem loop 3 are separated by at least one nucleotide, and/or
stem loop 3 and terminator segment are separated by at least one nucleotide.

34. The sgRNA of claim 1, wherein the sgRNA is 100 nucleotides, or about 100 nucleotides, in length

35. The sgRNA of claim 1, wherein the sgRNA comprises a N-Acetylgalactosamine (GalNAc) or a triantennary GalNAc on the 3′-end.

36. An oligomeric compound comprising a modified oligonucleotide according to the following formula:
(SEQ ID NO: 1)
NₘₛNₘₛNₘₛNₜNₜNₜNₜNₜNₜNₜNₜNₜNₜNₜNₜNₜNₜNₜNₜNₜ
GₜUₜUₜUₜUₜAₜGₜAₜGₘCₘUₘAₘGₘAₘAₘAₘUₘAₘGₘCₘAₜ
AₜGₜUₜUₜAₜAₜAₜAₜUₜAₜAₜGₜGₜCₜUₜAₜGₜUₜCₜCₜGₜ
UₜUₜAₜUₜCₜAₘAₘCₘUₘUₘGₘAₘAₘAₘAₘAₘGₘUₘGₘGₘCₘ
AₘCₘCₘGₘAₘGₘUₘCₘGₘGₘUₘGₘCₘUₘₛUₘₛUₘₛUₘ,
wherein:
A=an adenine nucleobase or an adenine nucleobase analogue,
C=a cytosine nucleobase or a cytosine nucleobase analogue,
G=a guanine nucleobase or a guanine nucleobase analogue,
U=an uracil nucleobase or an uracil nucleobase analogue,
t=a 2′-O-methyldithiomethyl (2′-O-MDTM) modified sugar moiety,
m=a 2′-O-methyl (2′-O-M) modified sugar moiety,
N=A, C, G, or U, and
s=a phosphorothioate (ps) internucleotide linkage.

37. (canceled)

38. (canceled)

39. The oligomeric compound of claim 36, wherein the adenine nucleobase analogue comprises 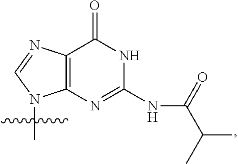
the cytosine nucleobase analogue comprises 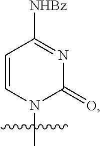
and the guanine nucleobase analogue comprises

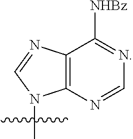

40. The oligomeric compound of claim 36, wherein a nucleobase analogue comprises 2-aminopurine, thymine, 2,6-diaminopurine, 2-pyrimidione, 5-methylcytosine, 5-hydroxymethylcytosine, hypoxanthine, xanthine, 7-methylguanine, or 5,6-dihydrouracil.

41. (canceled)

42. A composition comprising the sgRNA of claim 1 with the 2′-O-methyldithiomethyl groups of modified sugar moieties thereof converted to 2′-O-methanethiol groups by reduction of disulfide bonds of 2′-O-methyldithiomethyl groups and 2′-O-methanethiol groups converted to 2′-OH in complex with an RNA-guided endonuclease,
optionally wherein the RNA-guided endonuclease is a small Cas nuclease or a small RNA-guided endonuclease,
optionally wherein the RNA-guided endonuclease is a Cas9, a Cas12, aCas13, or a variant thereof,
optionally wherein the RNA-guided endonuclease is a Streptococcus pyogenes Cas9 (SpyCas9) or a Staphylococcus aureus (SaCas9),
optionally wherein the RNA-guided endonuclease is a variant of Cas9, and the variant of Cas9 is a small Cas9, a dead Cas9 (dCas9), or a Cas9 nickase, and/or
optionally wherein the RNA-guided endonuclease is a Streptococcus pyogenes Cas9 (SpyCas9).

43.-53. (canceled)

54. A method of treating a subject in need thereof, comprising:
administering the sgRNA of claim 1, or a composition thereof to the subject.

55. (canceled)


SEQUENCE LISTING (6 sequences)

SEQ ID NO: 1 - RNA, 100 nt, synthetic construct
  source 1..100: mol_type = other RNA
  misc_feature 1..20: note = N is A, U, G or C or an analogue thereof.
  modified_base 1..3: mod_base = OTHER; note = N has a 2'-O-methyl modified sugar moiety
  misc_feature 1..3: note = a phosphorothioate internucleotide linkage
  modified_base 4..20: mod_base = OTHER; note = N has a 2'-O-methyldithiomethyl modified sugar moiety.
  misc_feature 97..100: note = a phosphorothioate internucleotide linkage
  modified_base 68..100: mod_base = OTHER; note = 2'-O-methyl modified sugar moeity
  modified_base 29..40: mod_base = OTHER; note = 2'-O-methyl modified sugar moiety
  modified_base 21..28: mod_base = OTHER; note = 2'-O-methyldithiomethyl modified sugar moiety
  modified_base 41..67: mod_base = OTHER; note = 2'-O-methyldithiomethyl modified sugar moiety
nnnnnnnnnnnnnnnnnnnngttttagagctagaaatagcaagttaaaataaggctagtccgttatcaacttgaaaaagtggcaccgagtcggtgctttt

SEQ ID NO: 2 - RNA, 100 nt, synthetic construct; identical to SEQ ID NO: 1
  source 1..100: mol_type = other RNA
  modified_base 1..3: mod_base = OTHER; note = n is a 2'-O-methyl-adenosine, a 2'-O-methyl-cytodine, a 2'-O-methyl-guanosine, or a 2'-O-methyl-uradine.
  misc_feature 4..20: note = N is a 2'-O-methyldithiomethyl-adenosine, a 2'-O-methyldithiomethyl-cytodine, a 2'-O-methyldithiomethyl-guanosine, or a 2'-O-methyldithiomethyl-uradine.
  modified_base 21..28: mod_base = OTHER; note = 2'-O-methyldithiomethyl modified sugar moiety
  modified_base 29..40: mod_base = OTHER; note = 2'-O-methyl modified sugar moiety
  modified_base 41..67: mod_base = OTHER; note = 2'-O-methyldithiomethyl modified sugar moiety
  modified_base 68..100: mod_base = OTHER; note = 2'-O-methyl modified sugar moiety
  misc_feature 1..4: note = a phosphorothioate internucleoside linkage
  misc_feature 97..100: note = a phosphorothioate internucleoside linkage

SEQ ID NO: 3 - RNA, 100 nt, synthetic construct; identical to SEQ ID NO: 1
  source 1..100: mol_type = other RNA
  misc_feature 1..20: note = n is a, g, c or u.

SEQ ID NO: 4 - RNA, 20 nt, synthetic construct
  source 1..20: mol_type = other RNA
  misc_feature 1..4: note = a phosphorothioate internucleotide backbone linkage
gagaacgcaccactttacga

SEQ ID NO: 5 - RNA, 33 nt, synthetic construct
  source 1..33: mol_type = other RNA
  misc_feature 1..4: note = a phosphorothioate internucleotide linkage
  modified_base 1..3: mod_base = OTHER; note = 2'-O-methyl modified sugar moiety
  modified_base 4..28: mod_base = OTHER; note = 2'-O-methyldithiomethyl modified sugar moiety
  modified_base 29..33: mod_base = OTHER; note = 2'-O-methyl modified sugar moiety
gagaacgcaccactttacgagttttagagctag

SEQ ID NO: 6 - RNA, 39 nt, synthetic construct
  source 1..39: mol_type = other RNA
  modified_base 1..7: mod_base = OTHER; note = 2'-O-methyl modified sugar moiety
  modified_base 8..34: mod_base = OTHER; note = 2'-O-methyldithiomethyl modified sugar moiety
  modified_base 35..39: mod_base = OTHER; note = 2'-O-methyl modified sugar moiety
  misc_feature 1: note = 5' is attached to a phosphate.
aaatagcaagttaaaataaggctagtccgttatcaactt